SEQUENCE LISTING (134 sequences)

SEQ ID NO: 1 - protein, 14 aa, Homo sapiens
TTNIQGINLLFSSR

SEQ ID NO: 2 - protein, 18 aa, Homo sapiens
ILTVPGHLDEMQLDIQAR

SEQ ID NO: 3 - protein, 16 aa, Homo sapiens
ASAGLLGAHAAAITAY

SEQ ID NO: 4 - protein, 26 aa, Homo sapiens
VTASDPLDTLGSEGALSPGGVASLLR

SEQ ID NO: 5 - protein, 21 aa, Homo sapiens
VGLSGMAIADVTLLSGFHALR

SEQ ID NO: 6 - protein, 19 aa, Homo sapiens
DDPDAPLQPVTPLQLFEGR

SEQ ID NO: 7 - protein, 25 aa, Homo sapiens
ITPGKPYILTVPGHLDEMQLDIQAR

SEQ ID NO: 8 - protein, 26 aa, Homo sapiens
LLLFSPSVVHLGVPLSVGVQLQDVPR

SEQ ID NO: 9 - protein, 9 aa, Homo sapiens
AEFQDALEK

SEQ ID NO: 10 - protein, 9 aa, Homo sapiens
SCGLHQLLR

SEQ ID NO: 11 - protein, 12 aa, Homo sapiens
LNMGITDLQGLR

SEQ ID NO: 12 - protein, 26 aa, Homo sapiens
VTASDPLDTLGSEGALSPGGVASLLR

SEQ ID NO: 13 - protein, 9 aa, Homo sapiens
LELSVDGAK

SEQ ID NO: 14 - protein, 27 aa, Homo sapiens
LQETSNWLLSQQQADGSFQDPCPVLDR

SEQ ID NO: 15 - protein, 26 aa, Homo sapiens
LLLFSPSVVHLGVPLSVGVQLQDVPR

SEQ ID NO: 16 - protein, 10 aa, Homo sapiens
FGLLDEDGKK

SEQ ID NO: 17 - protein, 15 aa, Homo sapiens
HLVPGAPFLLQALVR

SEQ ID NO: 18 - protein, 12 aa, Homo sapiens
DFALLSLQVPLK

SEQ ID NO: 19 - protein, 13 aa, Homo sapiens
EELVYELNPLDHR

SEQ ID NO: 20 - protein, 24 aa, Homo sapiens
FQILTLWLPDSLTTWEIHGLSLSK

SEQ ID NO: 21 - protein, 26 aa, Homo sapiens
VTASDPLDTLGSEGALSPGGVASLLR

SEQ ID NO: 22 - protein, 19 aa, Homo sapiens
ALEILQEEDLIDEDDIPVR

SEQ ID NO: 23 - protein, 11 aa, Homo sapiens
VDVQAGACEGK

SEQ ID NO: 24 - protein, 9 aa, Homo sapiens
DHAVDLIQK

SEQ ID NO: 25 - protein, 14 aa, Homo sapiens
TTNIQGINLLFSSR

SEQ ID NO: 26 - protein, 18 aa, Homo sapiens
ILTVPGHLDEMQLDIQAR

SEQ ID NO: 27 - protein, 16 aa, Homo sapiens
ASAGLLGAHAAAITAY

SEQ ID NO: 28 - protein, 26 aa, Homo sapiens
VTASDPLDTLGSEGALSPGGVASLLR

SEQ ID NO: 29 - protein, 21 aa, Homo sapiens
VGLSGMAIADVTLLSGFHALR

SEQ ID NO: 30 - protein, 19 aa, Homo sapiens
DDPDAPLQPVTPLQLFEGR

SEQ ID NO: 31 - protein, 25 aa, Homo sapiens
ITPGKPYILTVPGHLDEMQLDIQAR

SEQ ID NO: 32 - protein, 26 aa, Homo sapiens
LLLFSPSVVHLGVPLSVGVQLQDVPR

SEQ ID NO: 33 - protein, 9 aa, Homo sapiens
AEFQDALEK

SEQ ID NO: 34 - protein, 9 aa, Homo sapiens
SCGLHQLLR

SEQ ID NO: 35 - protein, 12 aa, Homo sapiens
LNMGITDLQGLR

SEQ ID NO: 36 - protein, 26 aa, Homo sapiens
VTASDPLDTLGSEGALSPGGVASLLR

SEQ ID NO: 37 - protein, 13 aa, Homo sapiens
AEMADQAAAWLTR

SEQ ID NO: 38 - protein, 9 aa, Homo sapiens
LELSVDGAK

SEQ ID NO: 39 - protein, 26 aa, Homo sapiens
LLLFSPSVVHLGVPLSVGVQLQDVPR

SEQ ID NO: 40 - protein, 10 aa, Homo sapiens
FGLLDEDGKK

SEQ ID NO: 41 - protein, 15 aa, Homo sapiens
HLVPGAPFLLQALVR

SEQ ID NO: 42 - protein, 12 aa, Homo sapiens
DFALLSLQVPLK

SEQ ID NO: 43 - protein, 13 aa, Homo sapiens
EELVYELNPLDHR

SEQ ID NO: 44 - protein, 24 aa, Homo sapiens
FQILTLWLPDSLTTWEIHGLSLSK

SEQ ID NO: 45 - protein, 26 aa, Homo sapiens
VTASDPLDTLGSEGALSPGGVASLLR

SEQ ID NO: 46 - protein, 19 aa, Homo sapiens
ALEILQEEDLIDEDDIPVR

SEQ ID NO: 47 - protein, 11 aa, Homo sapiens
VDVQAGACEGK

SEQ ID NO: 48 - protein, 9 aa, Homo sapiens
DHAVDLIQK

SEQ ID NO: 49 - protein, 9 aa, Homo sapiens
EIGELYLPK

SEQ ID NO: 50 - protein, 12 aa, Homo sapiens
DEELSCTVVELK

SEQ ID NO: 51 - protein, 18 aa, Homo sapiens
HPNSPLDEENLTQENQDR

SEQ ID NO: 52 - protein, 18 aa, Homo sapiens
LYGSEAFATDFQDSAAAK

SEQ ID NO: 53 - protein, 18 aa, Homo sapiens
HPNSPLDEENLTQENQDR

SEQ ID NO: 54 - protein, 11 aa, Homo sapiens
ITLLSALVETR

SEQ ID NO: 55 - protein, 19 aa, Homo sapiens
AVLDVFEEGTEASAATAVK

SEQ ID NO: 56 - protein, 10 aa, Homo sapiens
ADLSGITGAR

SEQ ID NO: 57 - protein, 19 aa, Homo sapiens
AVLDVFEEGTEASAATAVK

SEQ ID NO: 58 - protein, 17 aa, Homo sapiens
GTHVDLGLASANVDFAF

SEQ ID NO: 59 - protein, 9 aa, Homo sapiens
EIGELYLPK

SEQ ID NO: 60 - protein, 12 aa, Homo sapiens
VVAGVANALAHK

SEQ ID NO: 61 - protein, 10 aa, Homo sapiens
LLVVYPWTQR

SEQ ID NO: 62 - protein, 19 aa, Homo sapiens
FFESFGDLSTPDAVMGNPK

SEQ ID NO: 63 - protein, 9 aa, Homo sapiens
CVLAHHFGK

SEQ ID NO: 64 - protein, 23 aa, Homo sapiens
QKPDGVFQEDAPVIHQEMIGGLR

SEQ ID NO: 65 - protein, 14 aa, Homo sapiens
IPIEDGSGEVVLSR

SEQ ID NO: 66 - protein, 13 aa, Homo sapiens
SSLSVPYVIVPLK

SEQ ID NO: 67 - protein, 20 aa, Homo sapiens
GQDLVVLPLSITTDFIPSFR

SEQ ID NO: 68 - protein, 23 aa, Homo sapiens
QKPDGVFQEDAPVIHQEMIGGLR

SEQ ID NO: 69 - protein, 14 aa, Homo sapiens
IPIEDGSGEVVLSR

SEQ ID NO: 70 - skipped

SEQ ID NO: 71 - protein, 20 aa, Homo sapiens
GQDLVVLPLSITTDFIPSFR

SEQ ID NO: 72 - protein, 22 aa, Homo sapiens
KPVDEYKDCHLAQVPSHTVVAR

SEQ ID NO: 73 - protein, 11 aa, Homo sapiens
EGYYGYTGAFR

SEQ ID NO: 74 - protein, 13 aa, Homo sapiens
SDNCEDTPEAGYF

SEQ ID NO: 75 - protein, 18 aa, Homo sapiens
HDTSLKPISVSYNPATAK

SEQ ID NO: 76 - protein, 12 aa, Homo sapiens
ADGLAVIGVLMK

SEQ ID NO: 77 - protein, 16 aa, Homo sapiens
LYPIANGNNQSPVDIK

SEQ ID NO: 78 - protein, 12 aa, Homo sapiens
VVAGVANALAHK

SEQ ID NO: 79 - protein, 10 aa, Homo sapiens
LLVVYPWTQR

SEQ ID NO: 80 - protein, 11 aa, Homo sapiens
LEGSSGGIGER

SEQ ID NO: 81 - protein, 8 aa, Homo sapiens
GNLKIHLR

SEQ ID NO: 82 - protein, 7 aa, Homo sapiens
AHVDALR

SEQ ID NO: 83 - protein, 25 aa, Homo sapiens
LQDGLLHITTCSFVAPWNSLSLAQR

SEQ ID NO: 84 - protein, 30 aa, Homo sapiens
KVADALTNAVAHVDDMPNALSALSDLHAHK

SEQ ID NO: 85 - protein, 18 aa, Homo sapiens
ILCEDPLPPIPKDSSRPK

SEQ ID NO: 86 - protein, 26 aa, Homo sapiens
PSSLTNLSSSSGMTSLSSVSGSVMSV

SEQ ID NO: 87 - protein, 26 aa, Homo sapiens
PSSLTNLSSSSGMTSLSSVSGSVMSV

SEQ ID NO: 88 - protein, 15 aa, Homo sapiens
SSSNEEVMFLTVQVK

SEQ ID NO: 89 - protein, 21 aa, Homo sapiens
GGTLSTPQTGSENDALYEYLR

SEQ ID NO: 90 - protein, 18 aa, Homo sapiens
CLPGFHMLTDAGCTQDQR

SEQ ID NO: 91 - protein, 13 aa, Homo sapiens
ITVVDALHEIPVK

SEQ ID NO: 92 - protein, 19 aa, Homo sapiens
NENGIDAEPAEEAVIQKPR

SEQ ID NO: 93 - protein, 16 aa, Homo sapiens
PESGEQAVAAVEALER

SEQ ID NO: 94 - protein, 21 aa, Homo sapiens
AFGGAAGGAGGGGGGGGGAGA

SEQ ID NO: 95 - protein, 13 aa, Homo sapiens
CNMKMELEQANER

SEQ ID NO: 96 - protein, 16 aa, Homo sapiens
KIVSDGNGMNAWVAWR

SEQ ID NO: 97 - protein, 12 aa, Homo sapiens
EDLDCTPGFQQK

SEQ ID NO: 98 - protein, 13 aa, Homo sapiens
QADTTQYVPMLER

SEQ ID NO: 99 - protein, 10 aa, Homo sapiens
LLVVYPWTQR

SEQ ID NO: 100 - protein, 28 aa, Homo sapiens
ITTGGDVINNGLWNMVSVEELEHSISIK

SEQ ID NO: 101 - protein, 11 aa, Homo sapiens
SSGQAGNKSER

SEQ ID NO: 102 - protein, 7 aa, Homo sapiens
MATMENK

SEQ ID NO: 103 - protein, 8 aa, Homo sapiens
FVGQGGAR

SEQ ID NO: 104 - protein, 23 aa, Homo sapiens
FINLKIMGESSLAPGTLPKPSVK

SEQ ID NO: 105 - protein, 11 aa, Homo sapiens
TLLSNLEEAKK

SEQ ID NO: 106 - protein, 15 aa, Homo sapiens
GYPGVQAPEDLEWER

SEQ ID NO: 107 - protein, 24 aa, Homo sapiens
GEDSSEEKHLEEPGETQNAFLNER

SEQ ID NO: 108-110 - skipped (3 entries)

SEQ ID NO: 111 - protein, 19 aa, Homo sapiens
  MOD_RES (15)..(15): Oxidized Methionine residue
FFESFGDLSTPDAVMGNPK

SEQ ID NO: 112 - protein, 17 aa, Homo sapiens
  MOD_RES (1)..(1): Oxidized Methionine residue
MCPQLQQYEMHGPEGLR

SEQ ID NO: 113 - protein, 9 aa, Homo sapiens
MFLSFPTTK

SEQ ID NO: 114 - protein, 9 aa, Homo sapiens
  MOD_RES (1)..(1): Oxidized Methionine residue
MFLSFPTTK

SEQ ID NO: 115 - protein, 9 aa, Homo sapiens
VIAVNEVGR

SEQ ID NO: 116 - protein, 10 aa, Homo sapiens
TLLSNLEEAK

SEQ ID NO: 117 - protein, 9 aa, Homo sapiens
EMSQEESTR

SEQ ID NO: 118 - protein, 16 aa, Homo sapiens
SCDNPYIPNGDYSPLR

SEQ ID NO: 119 - protein, 9 aa, Homo sapiens
DYFMPCPGR

SEQ ID NO: 120 - protein, 10 aa, Homo sapiens
DSGFQMNQLR

SEQ ID NO: 121 - protein, 10 aa, Homo sapiens
  MOD_RES (6)..(6): Oxidized Methionine residue
DSGFQMNQLR

SEQ ID NO: 122 - protein, 11 aa, Homo sapiens
LSPIYNLVPVK

SEQ ID NO: 123 - protein, 11 aa, Homo sapiens
LGADMEDVCGR

SEQ ID NO: 124 - protein, 11 aa, Homo sapiens
  MOD_RES (5)..(5): Oxidized Methionine residue
LGADMEDVCGR

SEQ ID NO: 125 - protein, 11 aa, Homo sapiens
MTVTDQVNCPK

SEQ ID NO: 126 - protein, 11 aa, Homo sapiens
  MOD_RES (1)..(1): Oxidized Methionine residue
MTVTDQVNCPK

SEQ ID NO: 127 - protein, 8 aa, Homo sapiens
EQLTPLIK

SEQ ID NO: 128 - protein, 14 aa, Homo sapiens
SFFSFLGEAFDGAR

SEQ ID NO: 129 - protein, 10 aa, Homo sapiens
SGAGTELSVR

SEQ ID NO: 130 - protein, 22 aa, Homo sapiens
KPVDEYKDCHLAQVPSHTVVAR

SEQ ID NO: 131 - protein, 11 aa, Homo sapiens
EGYYGYTGAFR

SEQ ID NO: 132 - protein, 13 aa, Homo sapiens
SDNCEDTPEAGYF

SEQ ID NO: 133 - protein, 12 aa, Homo sapiens
KSASDLTWDNLK

SEQ ID NO: 134 - protein, 18 aa, Homo sapiens
DGPWDSPALILELEDAVR

(19) United States
(12) Patent Application Publication
(10) Pub. No.: US 2012/0178637 A1
(43) Pub. Date: Jul. 12, 2012

(54) BIOMARKERS AND METHODS FOR DETECTING ALZHEIMER'S DISEASE

(75) Inventors: Viswanath Devanarayan, Souderton, PA (US); Melanie Joy Patterson, Highland Park, IL (US); Jeffrey F. Waring, Franklin, WI (US); David Witte, Vernon Hills, IL (US)

(73) Assignee: ABBOTT LABORATORIES, Abbott Park, IL (US)

(21) Appl. No.: 12/919,469

(22) PCT Filed: Jul. 7, 2010
(86) PCT No.: PCT/US2010/041257

§ 371 Date: Aug. 25, 2010

Related U.S. Application Data
(60) Provisional application No. 61/223,567, filed on Jul. 7, 2009.

(51) Int. Cl. C40B 30/04; C07K 2/00; C07K 14/47; G06F 19/00; C07K 14/705; C12N 9/12; C12N 9/88; C40B 40/04; C40B 40/06; C12N 9/04

(52) U.S. Cl. 506/9; 506/16; 530/300; 530/350; 435/190; 435/194; 435/232; 530/385; 506/15; 702/19

(57) ABSTRACT

Methods for classifying a test sample as indicative of Alzheimer's disease use protein and peptide biomarkers that are differentially expressed in the cerebral spinal fluid (CSF) of subjects with Alzheimer's disease relative to age-matched controls. The methods also use protein and peptide signatures indicative of Alzheimer's disease. Microarrays and kits for detecting the protein and peptide biomarkers in CSF samples can be used to classify Alzheimer's disease state from test samples.

### RELATED APPLICATION INFORMATION

This application claims the benefit of priority to U.S. provisional application No. 61/223,567, filed on Jul. 7, 2009, the entire contents of which are herein incorporated by reference.

### TECHNICAL FIELD OF THE INVENTION

The present invention relates generally to the protein and peptide biomarkers of disease, and more specifically to protein and peptide markers indicative of Alzheimer's disease.

### BACKGROUND OF THE INVENTION

Alzheimer's disease (AD) is a progressive brain disease with a huge cost to human patients and their families. AD is the most common form of dementia, a common term for memory loss and other cognitive impairments. The impact of AD is also a growing concern for governments due to the increasing number of elderly citizens at risk. No cure for AD is currently available, though a number of drug and non-drug based therapies for ameliorating the symptoms of AD are widely accepted. In general, drug treatments for AD are directed at slowing the progression of symptoms. While many such drug treatments have proven effective for many patients, success is directly correlated with detecting the presence of disease at its earliest stages.

Currently, no biochemical tests are known for the diagnosis of AD or for monitoring the progression of the disease. Certain publications have identified proteins or signatures that could be used as diagnostic tools for AD (see, e.g., Gomez Ravetti, M. et al., PLoS One, 3e3111 (2008); and Shaw, L. M. et al., Ann Neurol, 65, 403-13 (2009)). Most AD biomarker studies are focused on the quantitative changes in tau and Aβ proteins and modifications of these proteins in the cerebral spinal fluid (CSF) from AD patients. These studies have led to a consensus that an increase in total and p-tau and a concomitant decrease in Aβ1-42 in CSF may be indicative of AD. However, these changes in t-tau, p-tau, Aβ1-42 are not specific indicators of AD and also occur in some other forms of dementia (N. Andreasen et al., Arch Neurol. 58, 373-379 (2001); Formichi, P. et al., J. Cell. Physiol. 208, 39-46 (2006); Lewczuk P, et al., Neurobiol. Aging. 25, 273-281 (2004); Sunderland T. et al., JAMA 289, 2094-2103 (2003); Bailey P. Can. J. Neurol. Sci. 34, Suppl. 1 S72-S76 (2007); Blennow K., J. Am. Soc. Exp. Neurotherapeutics. 1, 213-225 (2004)).

The global prevalence of AD is expected to grow from approximately 6 billion people in 2008 to 11 billion in 2030, and an urgent need exists to identify markers for early detection of AD and to monitor the effectiveness of potential new therapies. As the only body fluid in direct contact with the brain, cerebrospinal fluid (CSF) is a potentially rich source of molecular markers that may be able to provide early and specific indication of neurological disorders including AD.

### SUMMARY OF THE INVENTION

The present disclosure is based in part on the identification of proteins and peptides in cerebral spinal fluid (CSF) that surprisingly have been found to be differentially expressed in subjects known to have AD.

Accordingly, in one aspect, the present disclosure provides a method of classifying Alzheimer's disease state of a subject, comprising: a) providing a test sample from the subject; b) determining expression levels in the test sample of at least one protein or peptide biomarker selected from any of the biomarkers set out in TABLES 2A, 2B or 5, or determining expression levels in the test sample of the proteins or peptides comprising any one of the biomarker combinations set out in TABLES 3B, 3C, 4B, or 4C; c) classifying the levels of expression of the selected biomarkers relative to expression levels of the biomarkers in a reference tissue sample as altered or not altered; and d) classifying the test sample according to (c), wherein altered expression levels of the biomarkers in the tissue sample relative to expression levels of the biomarkers in the reference sample indicate a classification of Alzheimer's disease (AD) in the subject. The tissue sample may comprises a spinal fluid sample. The biomarkers may consist of at least one biomarker selected from the biomarkers set forth in Table 2A or in Table 2B, at least two of the biomarkers, or all of the biomarkers set forth in Table 2A or 2B. The biomarkers may consist of an optimal set of biomarkers as set forth in any one of Tables 3B, 3C, 4B or 4C. The biomarkers may consist of at least one, at least two for all the biomarkers as set forth in Table 5.

In another aspect, the present disclosure provides a method for classifying Alzheimer's disease (AD) state of a subject, comprising: a) selecting a statistically relevant multi-analyte panel from fluid samples obtained from human subjects including a control cohort consisting of healthy subjects and an AD cohort consisting of subjects diagnosed with AD, in which panel a plurality of protein or peptide biomarkers are differentially expressed to provide expression values for a reference AD panel and a control panel; b) conducting a Random Forests or Simulated Annealing analysis on the multi-analyte data from step (a) to derive a signature; c) applying a classification algorithm to the signature of step (b) to refine the signature; d) obtaining a test fluid sample from the subject; e) determining expression level in the test sample for each of the protein biomarkers used to specify the panel of (a); f) providing the results of step (e) to the classification model on the signature obtained from step (c) to obtain an output; and g) determining the classification of the disease state according to the output of step f), wherein the classification is either AD or control. In the method, the classification algorithm in (c) may be selected from: Linear Discriminant Analysis (LDA), Diagonal Linear Discriminant Analysis (DLDA), Diagonal Quadratic Discriminant Analysis (DQDA), Random Forests, Support Vector Machines, Neural Network, and k-Nearest Neighbor method. In the method, the multi-analyte panel may consist of an optimal panel as set forth in Table 3B, which may further have at least 72% sensitivity and at least 71% specificity for Alzheimer's disease. In the method, the multi-analyte panel may consist of an optimal panel as set forth in Table 3C, which further may have at least 60% sensitivity and at least 80% specificity for Alzheimer's disease. Alternatively, the multi-analyte panel may consist of an optimal panel as set forth in Table 4B, which may further have at least 78% sensitivity and at least 90% specificity for Alzheimer's disease. Alternatively, the multi-analyte panel may consist of an optimal panel as set forth in Table 4C, which may further have at least 76% sensitivity and at least 90% specificity for Alzheimer's disease.

In another aspect, the present disclosure provides a computer-implemented method for classifying a test sample obtained from a subject, comprising: (a) obtaining a dataset associated with the test sample, wherein the obtained dataset comprises quantitative data for at least one protein or peptide biomarker selected from any of the biomarkers set out in TABLES 2A, 2B or 5, or the obtained dataset comprises quantitative data for the biomarkers comprising any one of the biomarker combinations as set out in TABLES 3B, 3C, 4B, or 4C; (b) inputting the obtained dataset into an analytical process on a computer that compares the obtained dataset against one or more reference datasets; and (c) classifying the test sample according to the output of the analytical process, wherein the classification is selected from the group consisting of an Alzheimer's disease (AD) classification and a normal classification. In the method, the test sample may be spinal fluid. The method may further comprise, after classification of the test sample, determining efficacy of a drug treatment in a clinical trial. The analytical process of (b) may further comprise application of a predictive model that comprises the one or more reference datasets. The one or more reference datasets may comprise quantitative data obtained from one or more human subjects selected from a group consisting of healthy subjects and subjects diagnosed with AD. In the method, the protein or peptide biomarkers comprise an optimal panel selected from a multi-analyte panel consisting of any one of the biomarker combinations set out in TABLES 3B, 3C, 4B, or 4C. In the method, the analytical process may comprise applying to the obtained dataset either Random Forests or Simulated Annealing algorithm to derive optimal signatures, and applying at least one algorithm selected from: Linear Discriminant Analysis (LDA), Diagonal Linear Discriminant Analysis (DLDA), Diagonal Quadratic Discriminant Analysis (DQDA), Support Vector Machines, Neural Network, and k-Nearest Neighbor method to fit the classification model on the optimal signatures. In another aspect, the present disclosure provides a computer system comprising: (a) a database containing information identifying the expression level in spinal fluid of a set of genes encoding at least one protein or peptide biomarkers set out in any one of TABLES 2A, 2B, 3B, 3C, 4B, 4C and 5; and b) a user interface to view the information. In the computer system, the database further may comprise sequence information for the proteins. The database further comprises information identifying an expression level for each of the proteins in normal tissue. The database further comprises information identifying the expression level for the genes in tissue from a human subject diagnosed with AD.

In another aspect, the present disclosure provides a kit for classifying a test sample obtained from a human subject, comprising reagents for detecting at least one protein or peptide biomarkers selected from any one of the biomarkers set out in TABLES 2A, 2B or 5, or reagents for detecting any one of the protein or peptide biomarker combinations as set out in any one of TABLES 3B, 3C, 4B, or 4C. The biomarkers may consist of at least one or at least two biomarkers selected from the biomarkers set forth in Table 2A, or from the biomarkers set forth in Table 2B. Alternatively, the biomarkers may consist of an optimal set of biomarkers as set forth in any one of Tables 3B, 3C, 4B or 4C. The biomarkers may instead consist of at least one biomarker selected from the biomarkers set forth in Table 5, or at least two biomarkers selected from the biomarkers as set forth in Table 5, or all the biomarkers as set forth in Table 5. In any kit, the reagents can be antibodies.

In another aspect, the present disclosure provides a biomarker indicative of AD selected from any one of Tables 2A, 2B, 3B, 3C, 4B, 4C and 5. A plurality of biomarkers may be combined in an optimal panel as set forth in any one of Tables 3B, 3C, 4B and 4C.

In another aspect, the present disclosure provides an array of primers or probes for classifying one or more test samples for Alzheimer's disease state, the array comprising: at least two different primers or probes coupled to a solid support; wherein each primer or probe is capable of specifically hybridizing under stringent conditions to a protein or peptide biomarker selected from any of the biomarkers indicative of AD as set out in TABLES 2A, 2B, 3B, 3C, 4B, 4C or 5. In the array, the different primers or probes may consist of a minimum number of different primers or probes needed to specifically hybridizing under stringent conditions to each protein or peptide biomarker in each biomarker combination as set forth in any one of TABLES 3A, 3B, 4A and 4C. Alternatively, the biomarkers may be any one or more biomarkers selected from TABLES 2A and 2B having an altered expression level of each biomarker between the AD disease state and control that is at a q-value of <0.1. The biomarkers may be any one or more biomarkers selected from TABLES 2A, 2B and 5, wherein an altered expression level of each biomarker between the AD disease state and control is at a p-value of <0.05.

In another aspect, the present disclosure provides an isolated peptide having an amino acid sequence selected from the group consisting of SEQ ID NO: 111, SEQ ID NO: 112, SEQ ID NO: 114, SEQ ID NO: 121, SEQ ID NO: 124, and SEQ ID NO: 126.

### BRIEF DESCRIPTION OF THE DRAWINGS

FIG. 1 is a panel of plots showing a representative example of a protein (isoform A of GC-rich sequence) that was identified as being differentially expressed in AD versus control CSF samples. (A) Standard error chart, showing the average intensity in the AD versus control groups. (B) Variability chart showing the three injections in individual CSF samples across the AD and control groups.

FIG. 2 is a heatmap showing the pattern of significant protein changes across individual AD CSF samples relative to combined controls. Boxes shown in green are downregulated in AD relative to control, boxes in red are upregulated in AD relative to control and boxes in white are not changed relative to controls.

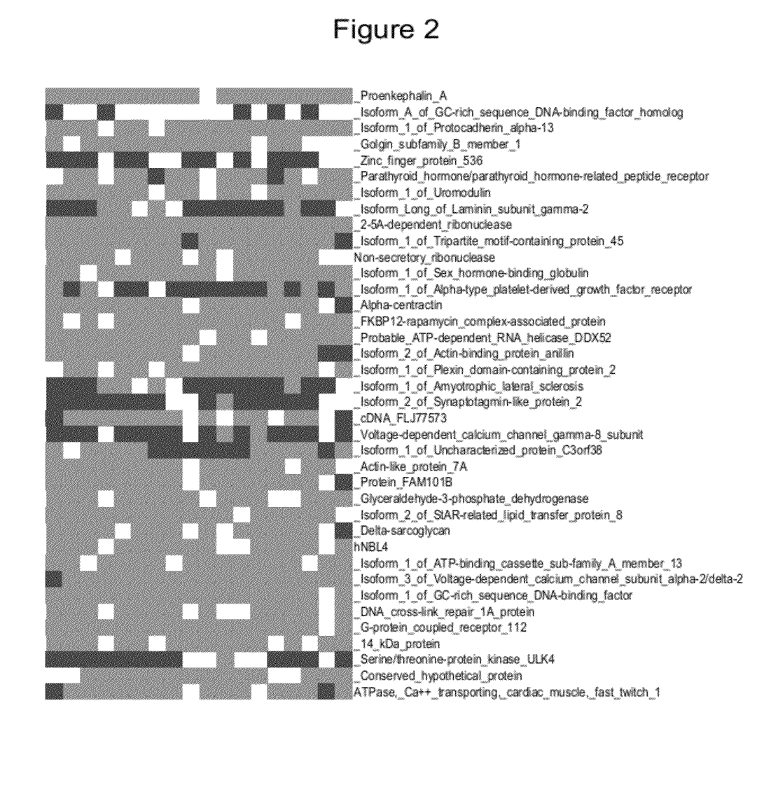

FIG. 3 is a heatmap showing the relative changes for proteins identified as being significantly regulated across the longitudinal AD CSF samples. Boxes shown in green are downregulated in AD relative to control, boxes in red are upregulated in AD relative to control and boxes in white are not changed relative to controls.

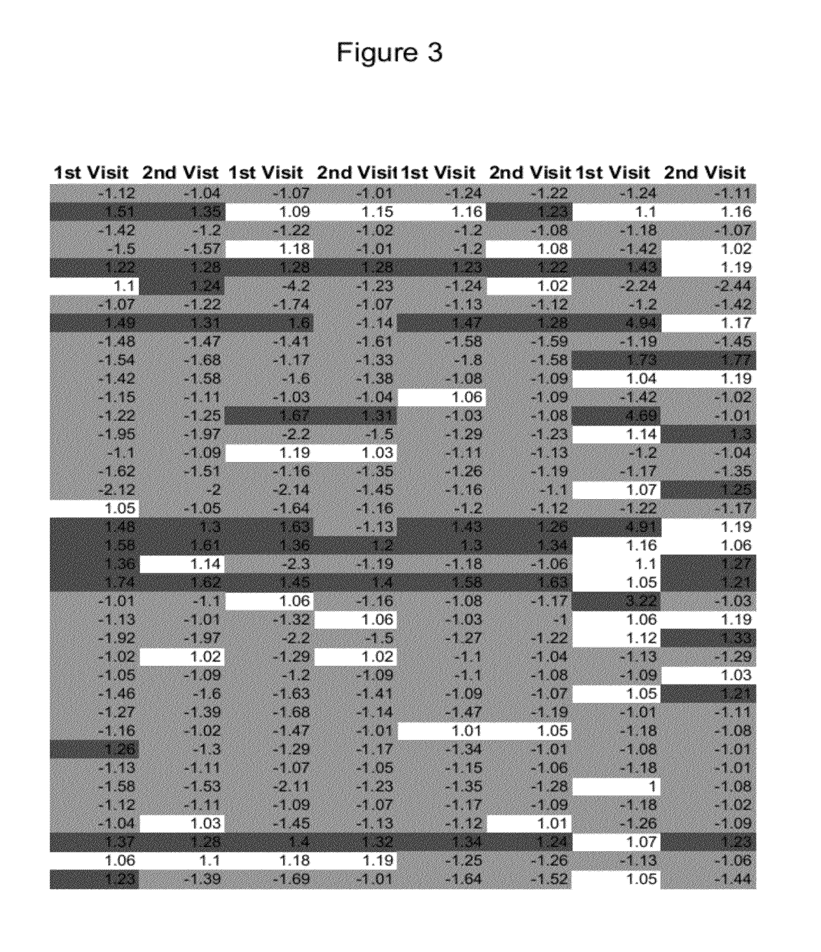

FIG. 4A is a panel of plots showing the average expression levels for the ten (10) proteins identified in the first protein signature.

FIG. 4B is a panel of plots showing the expression levels for the fifteen (15) proteins identified in the second protein signature.

FIG. 5A is a panel of plots showing the average expression levels for the six (6) peptides identified in the first peptide signature.

FIG. 5A is a panel of plots showing the expression levels for the eight (8) peptides identified in the second peptide signature analysis.

FIG. 6 is a bar graph of average number of unique spectra per protein, for fifteen selected proteins, with non-overlapping error bars.

FIG. 7 is a plot of a one-way ANOVA of the change in Log2Area between pooled AD samples and control samples for Alpha_{—}2_Macroglobulin.

FIG. 8 is a plot of a one-way ANOVA of the change in Log2Area between pooled AD samples and control samples for ApoA1.

FIG. 9 is a plot of a one-way ANOVA of the change in Log2Area between pooled AD samples and control samples for ApoAII.

FIG. 10 is a plot of a one-way ANOVA of the change in Log2Area between pooled AD samples and control samples for ApoD.

FIG. 11 is a plot of a one-way ANOVA of the change in Log2Area between pooled AD samples and control samples for ApoE, non-oxidized form.

FIG. 12 is a plot of a one-way ANOVA of the change in Log2Area between pooled AD samples and control samples for C3 fragment.

FIG. 13 is a plot of a one-way ANOVA of the change in Log2Area between pooled AD samples and control samples for C4B.

FIG. 14 is a plot of a one-way ANOVA of the change in Log2Area between pooled AD samples and control samples for C9b.

FIG. 15 is a plot of a one-way ANOVA of the change in Log2Area between pooled AD samples and control samples for Carbonic anhydrase.

FIG. 16 is a plot of a one-way ANOVA of the change in Log2Area between pooled AD samples and control samples for Clustrin.

FIG. 17 is a plot of a one-way ANOVA of the change in Log2Area between pooled AD samples and control samples for Complement 4A.

FIG. 18 is a plot of a one-way ANOVA of the change in Log2Area between pooled AD samples and control samples for Complement H.

FIG. 19 is a plot of a one-way ANOVA of the change in Log2Area between pooled AD samples and control samples for FKBP12.

FIG. 20 is a plot of a one-way ANOVA of the change in Log2Area between pooled AD samples and control samples for Hemoglobin alpha.

FIG. 21 is a plot of a one-way ANOVA of the change in Log2Area between pooled AD samples and control samples for Hemoglobin subunit beta.

FIG. 22 is a plot of a one-way ANOVA of the change in Log2Area between pooled AD samples and control samples for Hemopexin.

FIG. 23 is a plot of a one-way ANOVA of the change in Log2Area between pooled AD samples and control samples for LAMC2.

FIG. 24 is a plot of a one-way ANOVA of the change in Log2Area between pooled AD samples and control samples for Metalloproteinase inhibitor 1.

FIG. 25 is a plot of a one-way ANOVA of the change in Log2Area between pooled AD samples and control samples for NCAM.

FIG. 26 is a plot of a one-way ANOVA of the change in Log2Area between pooled AD samples and control samples for Secretogranin 1

FIG. 27 is a plot of a one-way ANOVA of the change in Log2Area between pooled AD samples and control samples for Serrotransferrin, non-oxidized form.

FIG. 28 is a plot of a one-way ANOVA of the change in Log2Area between pooled AD samples and control samples for SIRPG1.

FIG. 29 is a plot of a one-way ANOVA of the change in Log2Area between pooled AD samples and control samples for Tetranectin.

### DETAILED DESCRIPTION OF THE INVENTION

Section headings as used in this section and the entire disclosure herein are not intended to be limiting.

### A. Definitions

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the terms “subject” and “patient” are used interchangeably irrespective of whether the subject has or is currently undergoing any form of treatment. As used herein, the terms “subject” and “subjects” refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). Preferably, the subject is a human.

Unless otherwise defined herein, scientific and technical terms used in connection with the present disclosure shall have the meanings that are commonly understood by those of ordinary skill in the art. The meaning and scope of the terms should be clear; however, in the event of any latent ambiguity, definitions provided herein take precedent over any dictionary or extrinsic definition. Further, unless otherwise required by context, singular terms shall include pluralities and plural terms shall include the singular.

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Generally, nomenclatures used in connection with, and techniques of, cell and tissue culture, molecular biology, immunology, microbiology, genetics and protein and nucleic acid chemistry and hybridization described herein are those well known and commonly used in the art.

As used interchangeably herein, the terms “spinal fluid”, “cerebrospinal fluid” and “CSF” refer to that clear bodily fluid that occupies the subarachnoid space and the ventricular system around and inside the brain and spinal cord.

As used herein, the term “accuracy” refers to the overall ability of an individual marker or a composite of markers to correctly identify patients with the disease and patients without the disease. As used herein, the term “estimated effect of AD” refers to the estimated percentage change in a feature per year in the disease population. The current standard for dementia is a decrease of about 6% per year.

As used herein, the term “CERAD” refers to the Consortium to Establish a Registry for Alzheimer's Disease as recognized and used by health professionals studying or working with AD patients.

As used herein, the term “classifier” refers to any computational method that takes in a features as input and provides a class, such as for example “Alzheimer's disease” or “control”, as output.

As used herein, the terms “neural network”, Linear Discriminant Analysis (LDA), Diagonal Linear Discriminant Analysis (DLDA), Diagonal Quadratic Discriminant Analysis (DQDA), Support Vector Machines, Neural Network, and k-Nearest Neighbor method refer to statistical models for analyzing an input vector.

As used herein, the term “random forest” refers to a machine learning ensemble classifier developed by Leo Breiman and Adele Cutler, which consists of multiple single classification trees. (See, e.g., L. Breiman, Random Forests, MACHINE LEARNING 45 (1): 5-32. (2001)). To classify a new object from an input vector, the input vector is put down each of the trees in the forest, such that each tree gives a classification and “votes” for that class. The forest chooses the classification having the most votes (over all the trees in the forest).

As used herein, the term “test sample” generally refers to a biological material being tested for and/or suspected of containing an analyte of interest. The biological material may be derived from any biological source but preferably is a biological fluid likely to contain the analyte of interest, including but not limited to spinal fluid, stool, whole blood, serum, plasma, red blood cells, platelets, interstitial fluid, saliva, ocular lens fluid, cerebral spinal fluid, sweat, urine, ascites fluid, mucous, nasal fluid, sputum, synovial fluid, peritoneal fluid, vaginal fluid, menses, amniotic fluid, semen, soil, etc. Preferably, the test sample is spinal fluid. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. If such methods of pretreatment are employed with respect to the test sample, such pretreatment methods are such that the analyte of interest remains in the test sample at a concentration proportional to that in an untreated test sample (e.g., namely, a test sample that is not subjected to any such pretreatment method(s)).

As used herein, the term “sensitivity” refers to the ability of an individual marker or a composite of markers to correctly identify patients with a disease, e.g., Alzheimer's disease, which is the probability that the test is positive for a patient with the disease. For example, the current clinical criterion for AD is about 85% sensitive relative to autopsy confirmed cases in the best clinics. This number is usually much lower for patients in the earlier states of the disease, and varies considerably from clinic to clinic.

As used herein, the term “specificity” refers to the ability of an individual marker or a composite of markers to correctly identify patients that do not have the disease, i.e., the probability that the test is negative for a patient without disease. The current clinical criterion is that such marker(s) should provide a test that is at least 75% specific in the best clinics. This number is usually much lower for patients in the earlier states of the disease, and varies considerably from clinic to clinic.

As used herein, the term “AUC” refers to the area under the receiver operating characteristic (ROC) curve and refers to the overall ability of an individual marker or a composite of markers to correctly identify subjects with or without the disease.

As used herein, the term “signature” refers to a set of two or more proteins, genes, or peptides whose relative expression levels can be used to distinguish one or more groups with predetermined thresholds of sensitivity and specificity. An “optimal panel” of biomarkers is derived from a signature.

### B. Methods and Systems

The present disclosure is based in part on the surprising finding that certain proteins or peptides in cerebral spinal fluid are differentially expressed in subjects with Alzheimer's disease relative to age-matched controls. These proteins were also analyzed using the Neural Network and random-forest signature derivation method to identify representative signatures that display relatively high sensitivity and specificity for separating subjects with AD from controls. These proteins and peptides thus serve as biomarkers for classifying test samples, diagnostics or therapeutic monitoring, either individually or in a panel of biomarkers.

A biomarker for AD is any protein or peptide marker that can be found and measured in a test sample from a subject, such as a CSF sample, the expression level of which in the sample, in comparison to the expression level of the marker in a reference (control sample), is correlated with a diagnosis of AD. AD diagnosis can be determined or confirmed according to any one or more known clinical standards such as the clinical neuropsychology or behavior assessments promulgated by CERAD as known as recognized and used by health professionals. As described herein, the protein and peptide biomarkers as set forth in Tables 2A, 2B, 2C, 3B, 3C, 4B, 4C and 5 are characterized by one or both of the following: 1) on an individual basis, the expression level of the biomarker in an AD subject is significantly different from that in an age-matched control sample, and 2) the change in expression level of the biomarker in an AD subject relative to age-matched control, is significant as an element of a biomarker signature consisting of multiple biomarkers, which together establish a pattern of change in expression levels that is indicative of AD in a subject as compared to the pattern of expression observed for the same biomarkers in an age-matched control sample. Also of particular interest are biomarkers such as those set forth in TABLES 2A, 2B and 5, wherein each biomarker demonstrates an altered expression level of each biomarker between the AD disease state and control that is at a q-value of <0.1, or an altered expression level of each biomarker between the AD disease state and control is at a p-value of <0.05.

In the methods, to classify a test sample as AD positive, or a subject as having AD, the expression level of at least one of the biomarkers is obtained. It will be understood that any number of individually significant biomarkers, for example any one or more of those listed in Tables 2A, 2B, 2C and 5, can be used, including but not limited to one, two, three, four, five, six, seven, eight, nine, ten, fifteen, twenty, twenty-five, thirty, thirty-five, forty, forty-five, fifty, sixty, seventy, eighty, ninety and one hundred or more. For example, a total of 118 protein and peptide biomarkers (as listed in tables 2C and 5) are shown to be individually insignificant with respect to a classification or diagnosis of AD, and any subset of that 118 or all of those 118 may be used in any of the methods. Changes in expression level that are known to be significant between AD subjects and control subjects are considered indicative of AD.

Thus, for each marker, a reference or control expression level is established in control subjects to provide a reference or control level against which expression level(s) of the biomarker or biomarkers can be compared. More specifically, as described elsewhere herein, an expression level of any one or more biomarkers or any two or more biomarkers selected from any of TABLES 2A, 2B, 2C and 5 in a test sample can be determined and compared to a reference or control level for that biomarker.

Typically the level of each marker in a test sample from a subject is determined using an immunohistochemistry or immunoassay technique, such as for example an enzyme immunoassay (EIA), and for which kits are readily commerically available from a number of commercial suppliers. Alternatively, hybridization techniques including PCR or a mass spectrometric platform may be used to determine the level of each marker in a test sample. An exemplary microparticle enzyme immunoassay technology is the ARCHITECT® System available from Abbott Laboratories. The assay may involve a multiplex technique so the levels of two or more markers can be determined from the output of a single assay process. The marker level of any two or more of the biomarkers in a test sample can be combined to produce a marker signature (sometimes referred to as a “biomarker profile”), which is characterized by a pattern composed of at least of the two or more marker levels. An exemplary such pattern is composed of, for example, the biomarker combinations as set forth Tables 3B, 3C, 4B and 4C. With respect to a test sample, a marker signature having a predetermined pattern, i.e., satisfying certain criteria such as minimum fold changes in expression level between AD and control samples, is indicative of AD relative to a marker signature lacking the predetermined pattern.

Analysis of the marker levels may further involve comparing the levels of at least one or two markers with levels of the same markers in a control sample, which may be performed by applying a classification tree analysis. Classification tree analyses are generally well-known and can be readily applied to analysis of marker levels using a computer process. For example, a reference 3D contour plot can be generated that reflects the marker levels as described herein that correlate with a disease classification of AD. For any given subject, a comparable 3D plot can be generated and the plot compared to the reference 3D plot to determine whether the subject has a marker signature indicative of AD. Classification tree analyses are well-suited for analyzing marker levels because they are especially amenable to graphical display and are easy to interpret. It will however be understood that any computer-based application can be used that compares multiple marker levels from two different subjects, or from a reference sample and a subject, and provides an output that indicates a disease classification of AD as described herein.

The biomarkers may also be used to monitor the response of a subject or subjects to a drug treatment for AD. The monitoring can be validated or validated by numerous pathological, clinical and imaging methods such as those generally well known in the medical field, including ultrasound, CT and MRI.

It will also be understood that the methods can further involve obtaining the test sample from the subject using any tissue sampling technique including but not limited to lumbar puncture, cisternal puncture, fluoroscopy, myelogram, shunt, ventricular puncture, ventricular drain, or any combination thereof.

The methods can be used to classify one or more subjects, each subject having or suspected of having AD, for AD disease state or for efficacy of administration of an AD drug treatment. Such an approach involves determining, in a CSF sample from each subject, the expression level of at least one of the biomarkers and comparing the level of each marker to its level in a reference sample. Accordingly, based in part on the identification of these proteins as described in detail herein, a method for a method of classifying Alzheimer's disease state of a subject includes a) providing a test sample from the subject; b) determining expression levels in the test sample of at least one protein or peptide biomarker selected from any of the biomarkers set out in TABLES 2A, 2B or 5, or determining expression levels in the test sample of the proteins or peptides comprising any one of the biomarker combinations set out in TABLES 3B, 3C, 4B, or 4C; c) classifying the levels of expression of the selected biomarkers relative to expression levels of the biomarkers in a reference tissue sample as altered or not altered; and d) classifying the test sample according to (c), wherein altered expression levels of the biomarkers in the tissue sample relative to expression levels of the biomarkers in the reference sample indicate a classification of Alzheimer's disease (AD) in the subject.

The biomarkers may consist of one or more biomarkers selected from the biomarkers set forth in Table 2A or in Table 2B, or all of the biomarkers set forth in Table 2A or 2B. The biomarkers may consist of an optimal set of biomarkers as set forth in any one of Tables 3B, 3C, 4B or 4C. The biomarkers may consist of one or more biomarkers selected from the biomarkers set forth in Table 5. The biomarkers may consist of all the biomarkers as set forth in Table 5.

Biomarker signatures consisting of a multi-analyte panel of several biomarkers may also be derived and used. For example, a method for classifying Alzheimer's disease (AD) state of a subject may include: a) selecting a statistically relevant multi-analyte panel from fluid samples obtained from human subjects including a control cohort consisting of healthy subjects and an AD cohort consisting of subjects diagnosed with AD, in which panel a plurality of protein or peptide biomarkers are differentially expressed to provide expression values for a reference AD panel and a control panel; b) conducting a Random Forests or Simulated Annealing analysis on the multi-analyte data from step (a) to derive a signature; c) applying a classification algorithm to the signature of step (b) to refine the signature; d) obtaining a test fluid sample from the subject; e) determining expression level in the test sample for each of the protein biomarkers used to specify the panel of (a); e) comparing the results of step (e) to the signature obtained from step (c) to obtain an output; and f) determining the classification of the disease state according to the output of step e), wherein the classification is either AD or control. In the method, the classification algorithm in (c) may be selected from: Linear Discriminant Analysis (LDA), Diagonal Linear Discriminant Analysis (DLDA), Diagonal Quadratic Discriminant Analysis (DQDA), Random Forests, Support Vector Machines, Neural Network, and k-Nearest Neighbor method. In the method, the multi-analyte panel may consist of an optimal panel as set forth in Table 3B, which may further have at least 72% sensitivity and at least 71% specificity for Alzheimer's disease. Such a panel can be selected for example using the Neural Network algorithm and RF.imp signature derivation method as described in detail in the Examples and set forth in Table 3A, signature number 1. In the method, the multi-analyte panel may alternatively consist of an optimal panel as set forth in Table 3C, which further may have at least 60% sensitivity and at least 80% specificity for Alzheimer's disease. Such a panel can be selected for example using the Random Forest algorithm and Simulated Annealing signature derivation method as described in detail in the Examples and set forth in Table 3A, signature number 2. Alternatively, the multi-analyte panel may consist of an optimal panel as set forth in Table 4B, which may further have at least 78% sensitivity and at least 90% specificity for Alzheimer's disease. Such a panel can be selected for example using the Neural Network algorithm and RF.imp signature derivation method as described in detail in the Examples and set forth in Table 4A, signature number 1. Alternatively, the multi-analyte panel may consist of an optimal panel as set forth in Table 4C, which may further have at least 76% sensitivity and at least 90% specificity for Alzheimer's disease. Such a panel can be selected for example using the Neural Network algorithm and RF.imp signature derivation method as described in detail in the Examples and set forth in Table 4A, signature number 2.

Any of the methods may be implemented on a computer system. For example, further provided is a computer-implemented method for classifying a test sample obtained from a subject, which comprises: (a) obtaining a dataset associated with the test sample, wherein the obtained dataset comprises quantitative data for at least one protein or peptide biomarkers selected from any of the biomarkers set out in TABLES 2A, 2B or 5, or the obtained dataset comprises quantitative data for the biomarkers comprising any one of the biomarker combinations as set out in TABLES 3B, 3C, 4B, or 4C; (b) inputting the obtained dataset into an analytical process on a computer that compares the obtained dataset against one or more reference datasets; and (c) classifying the test sample according to the output of the analytical process, wherein the classification is selected from the group consisting of an Alzheimer's disease (AD) classification and a normal classification. The method may further comprise, after classification of the test sample, determining efficacy of a drug treatment in a clinical trial. The analytical process of (b) may further comprise application of a predictive model that comprises the one or more reference datasets. The one or more reference datasets may comprise quantitative data obtained from one or more human subjects selected from a group consisting of healthy subjects and subjects diagnosed with AD. In the method, the protein or peptide biomarkers comprise an optimal panel selected from a multi-analyte panel consisting of any one of the biomarker combinations set out in TABLES 3B, 3C, 4B, or 4C. The analytical process may comprise applying to the obtained dataset at least one algorithm selected from: Random Forests, Simulated Annealing algorithm, Linear Discriminant Analysis (LDA), Diagonal Linear Discriminant Analysis (DLDA), Diagonal Quadratic Discriminant Analysis (DQDA), Support Vector Machines, Neural Network, and k-Nearest Neighbor method.

A computer-implemented method may be used for determining differential expression of a multiplicity of gene transcripts of at least two subjects. For example, the computer-implemented method comprises the following steps: (a) providing a database comprising hybridization patterns that represent expression patterns of multiple genes for a plurality of subjects, wherein each hybridization pattern is generated by hybridizing an array of polynucleotide probes disclosed herein, with more than one labeled target polynucleotides corresponding to gene transcripts expressed in a distinct subject, wherein said hybridizing step yields detectable target-probe complexes with different levels of hybridization intensities; (b) receiving two or more of hybridization patterns for comparison; (c) determining differences in the selected hybridization patterns; and (d) displaying the results of said determination. The determining step includes the step of calculating the differences between the hybridization intensities of target-probe complexes localized in predetermined regions on the solid support.

Computer-implemented methods, for example for classifying a test sample obtained from a subject, use a computer system, which is configured to accept and analyze a data set of measurements of differential expression of a multiplicity of gene transcripts, such as may be indicated by a difference in expression signal. The expression signal may be based for example on mass spectroscopic analysis, immunoassay analysis, or hybridization patterns on an array of polynucleotide probes. Such a computer system may comprise, for example, (a) a database containing information identifying the expression level in spinal fluid of a set of genes encoding at least two proteins or peptide biomarkers set out in any one of TABLES 2A, 2B, 3B, 3C, 4B, 4C and 5; and b) a user interface to view the information. In the computer system, the database further may comprise sequence information for the genes. The database further comprises information identifying an expression level for each of the genes in normal tissue. The database further comprises information identifying the expression level for the genes in tissue from a human subject diagnosed with AD. The computer system may further include a search device for comparing the test expression level data to reference or control expression level data, and a retrieval device for obtaining the differences in expression levels.

Generally a computer-based system includes hardware and software. The database refers to memory, which can store test expression level data to reference or control expression level data, which are generated by mass spectroscopic analysis, immunoassay analysis, or hybridization. The data-storage device may also include a memory access device, which can access prerecorded array information. Non-limiting exemplary data storage devices are media storage, floppy drive, super floppy, tape drive, zip drive, syquest syjet drive, hard drive, CD Rom recordable (R), CD Rom rewritable (RW), M.D. drives, optical media, and punch cards/tape. A search device encompasses one or more programs which are implemented on the system to compare the test data to reference or control data, in order to detect the differences in expression levels. A variety of known algorithms are known and a variety of commercially available software is available for pattern recognition and can be used in computer-based systems. Examples of array analysis software include Biodiscovery, HP, and any of those applicable for image analyses. Search devices include those embodied in “Gene Array Scanner (Hewlett Packard)”, “General Scanning”, “reader Hitachi system”, “Genomics Solutions” and “GeneChip work station”. Finally, the retrieval device includes program(s), which are implemented on the system to retrieve the differences in expression levels detected by the search device. Hardware necessary for displaying the detected device may also form part of the retrieval device. The storage, search, retrieval devices may be assemble as any among well known devices including a PC, Mac, Cray, SGI machine, Sun machine, UNIX or LINUX based Workstations, Be OS systems, laptop computer, palmtop computer, and palm pilot system, or the like.

### C. Kits and Arrays

A kit for detecting AD or for monitoring AD in response to therapeutics such as but not limited to experimental therapeutics, may comprise materials for detecting the presence or level of at least two or more of the peptide or protein markers described herein. Alternatively, for example, a kit for classifying a test sample obtained from a subject, may comprise reagents for determining the expression level of at least one protein or peptide biomarker, or at least two biomarkersselected from any one of the biomarkers set out in TABLES 2A, 2B or 5, or reagents for determining the expression levels of the protein or peptide biomarker combinations as set out in any one of TABLES 3B, 3C, 4B, or 4C. It will be understood that reagents sufficient for determining the expression level(s) of any number of biomarkers may be included in the kit, as described above with respect to the methods. For example, the kit may include reagents sufficient for determining the expression level(s) of any one, two, three, four, five, six, seven, eight, nine, ten, fifteen, twenty, twenty-five, thirty, thirty-five, forty, forty-five, fifty, sixty, seventy, eighty, ninety or one hundred of the protein or peptide biomarkers. In any kit, the reagents can be antibodies. Alternatively, the kit may contain primers or probes as described herein below.

A kit can for example be used to practice any of the methods, such as a method for classifying a disease state of a subject, based on measurements of the expression levels of a single or multiple protein biomarkers in a test sample, after obtaining a test sample of CSF from the subject. For example, a kit may contain reagents for detecting the expression levels of the protein or peptide biomarkers using an immunoassay as described above. For example, FKBP12-rapamycin_complex-associated_protein (IPI00031410.1) expression levels could be measured directly from CSF samples (raw CSF without any manipulation following sample collection) using an ELISA or other sandwich-based immunoassay developed from antibodies as described above.

A kit may contain, for example, a solid support coated with one or more binding proteins such as antibodies, wherein each binding protein specifically binds to a protein or peptide biomarker listed in any of Tables 2A, 2B, 3B, 3C, 4B, 4C and 5. Such an antibody may function for example as a capture antibody. At least a second binding protein labeled with a detectable label may be used as a detection agent. It will be understood that such a kit may include reagents sufficient to perform multiplex analysis of expression levels of two or more of the protein or peptide biomarkers. A kit may also contain a control sample containing a predetermined reference or control level of each marker. Alternatively, a kit may include an array of two or more of the markers or truncated forms or fragments thereof.

A binding protein may be for example a polyclonal antibody, a monoclonal antibody, a chimeric antibody, a human antibody, an affinity maturated antibody or an antibody fragment. A sandwich immunoassay format may be used in which both a capture and a detection antibody are used for each marker. Antibodies may be bound, for example conjugated, to a detectable label. While monoclonal antibodies are highly specific to the marker/antigen, a polyclonal antibody can preferably be used as a capture antibody to immobilize as much of the marker/antigen as possible. A monoclonal antibody with inherently higher binding specificity for the marker/antigen may then preferably be used as a detection antibody for each marker/antigen. In any case, the capture and detection antibodies recognize non-overlapping epitopes on each marker, preferably without interfering with the binding of the other.

Polyclonal antibodies are raised by injecting (e.g., subcutaneous or intramuscular injection) an immunogen into a suitable non-human mammal (e.g., a mouse or a rabbit). Generally, the immunogen should induce production of high titers of antibody with relatively high affinity for the target antigen. If desired, the marker may be conjugated to a carrier protein by conjugation techniques that are well known in the art. Commonly used carriers include keyhole limpet hemocyanin (KLH), thyroglobulin, bovine serum albumin (BSA), and tetanus toxoid. The conjugate is then used to immunize the animal. The antibodies are then obtained from blood samples taken from the animal. The techniques used to produce polyclonal antibodies are extensively described in the literature (see, e.g., Methods of Enzymology, “Production of Antisera with Small Doses of Immunogen: Multiple Intradermal Injections,” Langone, et al. eds. (Acad. Press, 1981)). Polyclonal antibodies produced by the animals can be further purified, for example, by binding to and elution from a matrix to which the target antigen is bound. Those of skill in the art will know of various techniques common in the immunology arts for purification and/or concentration of polyclonal, as well as monoclonal, antibodies (see, e.g., Coligan, et al. (1991) Unit 9, Current Protocols in Immunology, Wiley Interscience).

For many applications, monoclonal antibodies (mAbs) are preferred. The general method used for production of hybridomas secreting mAbs is well known (Kohler and Milstein (1975) Nature, 256:495). Briefly, as described by Kohler and Milstein, the technique entailed isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody that bound to cancer cell lines. Confirmation of specificity among mAbs can be accomplished using routine screening techniques (such as the enzyme-linked immunosorbent assay, or “ELISA”) to determine the elementary reaction pattern of the mAb of interest. As used herein, the term “antibody” also encompasses antigen-binding antibody fragments, e.g., single chain antibodies (scFv or others), which can be produced/selected using phage display technology.

As those of skill in the art readily appreciate, antibodies can be also prepared by any of a number of commercial services (e.g., Berkeley Antibody Laboratories, Bethyl Laboratories, Anawa, Eurogenetec, etc.).

In kits according to the present disclosure, each binding protein may be bound to, i.e. immobilized on a solid phase. A solid phase can be any suitable material with sufficient surface affinity to bind an antibody, for example each capture antibody having a specific binding for one of the markers. The solid phase can take any of a number of forms, such as a magnetic particle, bead, test tube, microtiter plate, cuvette, membrane, a scaffolding molecule, quartz crystal, film, filter paper, disc or a chip. Useful solid phase materials include: natural polymeric carbohydrates and their synthetically modified, crosslinked, or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer. All of these materials may be used in suitable shapes, such as films, sheets, tubes, particulates, or plates, or they may be coated onto, bonded, or laminated to appropriate inert carriers, such as paper, glass, plastic films, fabrics, or the like. Nitrocellulose has excellent absorption and adsorption qualities for a wide variety of reagents including monoclonal antibodies. Nylon also possesses similar characteristics and also is suitable. Any of the above materials can be used to form an array, such as a microarray, of one or more specific binding reagents.

Alternatively, the solid phase can constitute microparticles. Microparticles useful in the present disclosure can be selected by one skilled in the art from any suitable type of particulate material and include those composed of polystyrene, polymethylacrylate, polypropylene, latex, polytetrafluoroethylene, polyacrylonitrile, polycarbonate, or similar materials. Further, the microparticles can be magnetic or paramagnetic microparticles, so as to facilitate manipulation of the microparticle within a magnetic field. In an exemplary embodiment the microparticles are carboxylated magnetic microparticles. Microparticles can be suspended in the mixture of soluble reagents and test sample or can be retained and immobilized by a support material. In the latter case, the microparticles on or in the support material are not capable of substantial movement to positions elsewhere within the support material. Alternatively, the microparticles can be separated from suspension in the mixture of soluble reagents and test sample by sedimentation or centrifugation. When the microparticles are magnetic or paramagnetic the microparticles can be separated from suspension in the mixture of soluble reagents and test sample by a magnetic field. The methods of the present disclosure can be adapted for use in systems that utilize microparticle technology including automated and semi-automated systems wherein the solid phase comprises a microparticle. Such systems include those described in pending U.S. App. No. 425,651 and U.S. Pat. No. 5,089,424, which correspond to published EPO App. Nos. EP 0 425 633 and EP 0 424 634, respectively, and U.S. Pat. No. 5,006,309.

Other considerations affecting the choice of solid phase include the ability to minimize non-specific binding of labeled entities and compatibility with the labeling system employed. For, example, solid phases used with fluorescent labels should have sufficiently low background fluorescence to allow signal detection. Following attachment of a specific capture antibody, the surface of the solid support may be further treated with materials such as serum, proteins, or other blocking agents to minimize non-specific binding.

Kits according to the present disclosure may include one or more detectable labels. The one or more specific binding reagents, e.g. antibodies, may be bound to a detectable label. Detectable labels suitable for use include any compound or composition having a moiety that is detectable by spectroscopic, photochemical, biochemical, immunochemical, electrical, optical, or chemical means. Such labels include, for example, an enzyme, oligonucleotide, nanoparticle chemiluminophore, fluorophore, fluorescence quencher, chemiluminescence quencher, or biotin. Thus for example, in an immunoassay kit configured to employ an optical signal, the optical signal is measured as an analyte concentration dependent change in chemiluminescence, fluorescence, phosphorescence, electrochemiluminescence, ultraviolet absorption, visible absorption, infrared absorption, refraction, surface plasmon resonance. In an immunoassay kit configured to employ an electrical signal, the electrical signal is measured as an analyte concentration dependent change in current, resistance, potential, mass to charge ratio, or ion count. In an immunoassay kit configured to employ a change-of-state signal, the change of state signal is measured as an analyte concentration dependent change in size, solubility, mass, or resonance.

Useful labels according to the present disclosure include magnetic beads (e.g., Dynabeads™), fluorescent dyes (e.g., fluorescein, Texas Red, rhodamine, green fluorescent protein) and the like (see, e.g., Molecular Probes, Eugene, Oreg., USA), chemiluminescent compounds such as acridinium (e.g., acridinium-9-carboxamide), phenanthridinium, dioxetanes, luminol and the like, radiolabels (e.g., 3H, 125I, 35S, 14C, or 32P), catalysts such as enzymes (e.g., horse radish peroxidase, alkaline phosphatase, beta-galactosidase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label can be attached to each antibody, for example to a detection antibody in a sandwich immunoassay format, prior to, or during, or after contact with the biological sample. So-called “direct labels” are detectable labels that are directly attached to or incorporated into the antibody prior to use in the assay. Direct labels can be attached to or incorporated into the detection antibody by any of a number of means well known to those of skill in the art.

In contrast, so-called “indirect labels” typically bind to each antibody at some point during the assay. Often, the indirect label binds to a moiety that is attached to or incorporated into the detection agent prior to use. Thus, for example, each antibody can be biotinylated before use in an assay. During the assay, an avidin-conjugated fluorophore can bind the biotin-bearing detection agent, to provide a label that is easily detected.

In another example of indirect labeling, polypeptides capable of specifically binding immunoglobulin constant regions, such as polypeptide A or polypeptide G, can also be used as labels for detection antibodies. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542). Such polypeptides can thus be labeled and added to the assay mixture, where they will bind to each capture and detection antibody, as well as to the autoantibodies, labeling all and providing a composite signal attributable to analyte and autoantibody present in the sample.

Some labels may require the use of an additional reagent(s) to produce a detectable signal. In an ELISA, for example, an enzyme label (e.g., beta-galactosidase) will require the addition of a substrate (e.g., X-gal) to produce a detectable signal. In an immunoassay kit configured to use an acridinium compound as the direct label, a basic solution and a source of hydrogen peroxide can also be included in the kit.

Test kits according to the present disclosure preferably include instructions for determining the level of each marker in a sample from the subject, for example by carrying out one or more immunoassays. The instructions may further include instructions for analyzing a test sample of a specific type, such as a blood sample, or more specifically a serum sample or a plasma sample. Instructions included in kits of the present disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Alternatively, nucleic acid primers or probes that specifically hybridize under stringent conditions to the protein or peptide biomarkers can be used in the methods according to conventional techniques of molecular biology, genomics and recombinant DNA, which are within the skill of the art. See, e.g., Sambrook, Fritsch and Maniatis, MOLECULAR CLONING: A LABORATORY MANUAL, 2 nd edition (1989); and CURRENT PROTOCOLS IN MOLECULAR BIOLOGY (F. M. Ausubel, et al. eds, (1987)).

A “probe” refers to a polynucleotide used for detecting or identifying its corresponding target polynucleotide in a hybridization reaction. A “primer” is a short polynucleotide, generally with a free 3′-OH group, that binds to a target or “template” potentially present in a sample of interest by hybridizing with the target, and thereafter promoting polymerization of a polynucleotide complementary to the target. The term “hybridize” as applied to a polynucleotide refers to the ability of the polynucleotide to form a complex that is stabilized via hydrogen bonding between the bases of the nucleotide residues in a hybridization reaction. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. The hybridization reaction may constitute a step in a more extensive process, such as the initiation of a PCR reaction, or the enzymatic cleavage of a polynucleotide by a ribozyme.

Hybridization can be performed under conditions of different stringency. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

The polynucleotide primers and probes can be obtained by chemical synthesis, recombinant cloning (PCR), or any combination thereof. Methods of chemical polynucleotide synthesis are well known in the art, as are methods of using the sequence data provided herein to obtain a desired polynucleotide by employing a DNA synthesizer, PCR machine, or ordering from a commercial service.

Selected primers or probes can be immobilized onto predetermined regions of a solid support by any suitable techniques that stably associate the primers or probes with the surface of a solid support, such that the polynucleotides remain localized to the predetermined region under hybridization and washing conditions. The polynucleotides can be covalently associated with or non-covalently attached to the support surface. Examples of non-covalent association include binding as a result of non-specific adsorption, ionic, hydrophobic, or hydrogen bonding interactions. Covalent association involves formation of chemical bond between the polynucleotides and a functional group present on the surface of a support. The functional may be naturally occurring or introduced as a linker. Non-limiting functional groups include but are not limited to hydroxyl, amine, thiol and amide. Exemplary techniques applicable for covalent immobilization of polynucleotide probes include, but are not limited to, UV cross-linking or other light-directed chemical coupling, and mechanically directed coupling as well known in the art.

Thus the primers or probes may be usefully provided in an array, such as a microarray. For example, an array of primers or probes for classifying one or more test samples for Alzheimer's disease state, may comprise at least two different primers or probes coupled to a solid support. Each primer or probe is capable of specifically hybridizing under stringent conditions to a protein or peptide biomarker selected from any of the biomarkers set out in TABLES 2A, 2B, 3B, 3C, 4B, 4C or 5. In the array, the different primers or probes may consist of a minimum number of different primers or probes needed to specifically hybridize under stringent conditions to each protein or peptide biomarker in each biomarker combination as set forth in any one of TABLES 3A, 3B, 4A and 4C. With the exception of the biomarker signatures including specific biomarker combinations, any number of biomarkers can be used, and thus any number of primers or probes can be included in array. For example, an array may be based on any two, three, four, five, six or more biomarkers selected from any of TABLES 2A, 2B and thus may include two, three, four, five, six or more different primers or probes. The array may be based on any two or more biomarkers selected from TABLES 2A and 2B and having an altered expression level of each biomarker between the AD disease state and control that is at a q-value of <0.1. Alternatively, the array may be based on any two or more biomarkers selected from TABLES 2A, 2B and 5, wherein an altered expression level of each biomarker between the AD disease state and control is significant at a p-value of <0.05.

A kit may contain one or more polynucleotide primer or probe arrays. Kits may allow simultaneous detection of the expression and/or quantification of the level of expression of multiple gene transcripts of a subject. Also encompassed are kits useful for detecting differential expression of a multiplicity of gene transcripts of a test subject in comparison to a control.

Each kit necessarily comprises the reagents needed for the hybridization procedure: an array of polynucleotide primers or probes used for detecting target polynucleotides; hybridization reagents that allow formation of stable target-primer or probe complexes during a hybridization reaction. The kits may also contain reagents useful for generating labeled target polynucleotides corresponding to gene transcripts of a test subject. Optionally, the arrays contained in the kits may be pre-hybridized with polynucleotides corresponding to gene transcripts of the control to which the test subject is compare.

Each reagent can be supplied in a solid form or dissolved/suspended in a liquid buffer suitable for inventory storage, and later for exchange or addition into the reaction medium when the test is performed. Suitable packaging is provided. The kit can optionally provide additional components that are useful in the procedure. These optional components include, but are not limited to, buffers, capture reagents, developing reagents, labels, reacting surfaces, means for detection, control samples, instructions, and interpretive information. The kits can be employed to test a variety of biological samples, including body fluid, solid tissue samples, tissue cultures or cells derived therefrom and the progeny thereof, and sections or smears prepared from any of these sources.

The present disclosure also encompasses isolated peptide markers having an oxidized methionine residue, which are indicative of AD. Specifically, the following amino acid sequences as set forth below in Table 5 are disclosed:

| | |
|---|---|
| FFESFGDLSTPDAVM*GNPK | (SEQ ID NO: 111) |
| M*CPQLQQYEMHGPEGLR | (SEQ ID NO: 112) |
| M*FLSFPTTK | (SEQ ID NO: 114) |
| DSGFQM*NQLR | (SEQ ID NO: 121) |
| LGADM*EDVCGR | (SEQ ID NO: 124) |
| M*TVTDQVNCPK | (SEQ ID NO: 126) |

### D. Adaptations of the Compositions and Methods of the Present Disclosure

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

### EXAMPLES

By way of example, and not of limitation, examples of the present disclosures shall now be given.

### Example 1

### Differentially Expressed Proteins in CSF of AD Subjects Relative to Age-Matched Controls

A global proteomics profiling study was conducted on CSF samples from 15 Alzheimer's patients and 10 age-matched control (AMC) subjects. In addition, 5 additional longitudinal AD CSF samples were analyzed after being obtained from a second visit, for a total of 20 AD subjects. Thus, thirty (30) human CSF samples were analyzed by Monarch Proteomics (10 AMC, 20 AD, Table 1).

Sample Preparation: The thirty CSF samples (20 Alzheimer's disease samples and 10 age-matched normal samples) were purchased from the PRECISIONMED Inc. (Detailed information in Table 1 herein above). Albumin and IgG were removed from the sample using Sigma Proteoprep spin columns. Resulting flow through fractions were denatured by 8 M urea, reduced by triethylphosphine, alkylated by iodoethanol, and digested by trypsin. (See Hale J E, Butler J P, Gelfanova V, You J S, Knierman M D (2004) A simplified procedure for the reduction and alkylation of cysteine residues in proteins prior to proteolytic digestion and mass spectral analysis Anal Biochem. 333 (1): 174-181).

Mass Spectrometric Analysis: Tryptic peptides (˜10 mg) were analyzed using Thermo-Fisher Scientific linear ion-trap mass spectrometer (LTQ) coupled with a Surveyor HPLC system (Thermo). C-18 reverse phase column (i.d.=2.1 mm, length=50 mm) was used to separate peptides with a flow rate of 200 mL/min. Peptides were eluted with a gradient from 5 to 45% acetonitrile developed over 120 min and data were collected in the triple-play mode (MS scan, zoom scan, and MS/MS scan. The acquired data were filtered, pooled and analyzed and database searches were conducted against the International Protein Index (IPI) human database and the non-Redundant-Homo Sapiens database (V3.85) and non-Redundant-Homo Sapiens database using both the X!Tandem and SEQUEST algorithms.

Protein quantification was carried out using a proprietary protein quantification algorithm licensed from Eli Lilly and Company (Can, S. et al., Mol Cell Proteomics, 3, 531-3 (2004)). Briefly, once the raw files were acquired from the LTQ, all extracted ion chromatograms (XIC) were aligned by retention time. To be used in the protein quantification procedure, each aligned peak must match precursor ion, charge state, fragment ions (MS/MS data) and retention time (within a one-minute window). After alignment, area-under-the-curve (AUC) for each individually aligned peak from each sample was measured, and these were compared for relative abundance. All peak intensities were transformed to a log2 scale before quantile normalization (Higgs, R E, et al., Journal of Proteome Research, Vol. 6, pp. 1758-1767 (2007)). Quantile normalization is a method of normalization that essentially ensures that every sample has a peptide intensity histogram of the same scale, location and shape. This normalization removes trends introduced by sample handling, sample preparation, total protein differences and changes in instrument sensitivity while running multiple samples. If multiple peptides have the same protein identification, then their quantile normalized log2 intensities were averaged to obtain log2 protein intensities. The log2 protein intensity is the final quantity that is analyzed statistically for each protein in the univariate and multivariate analysis.

Mass Spectrometric Analysis Tryptic peptides (˜10 μg) were analyzed using Thermo-Fisher Scientific linear ion-trap mass spectrometer (LTQ) coupled with a Surveyor HPLC system (Thermo). C-18 reverse phase column (i.d.=2.1 mm, length=50 mm) was used to separate peptides with a flow rate of 200 μL/min. Peptides were eluted with a gradient from 5 to 45% acetonitrile developed over 120 min and data were collected in the triple-play mode (MS scan, zoom scan, and MS/MS scan). The acquired data were filtered and analyzed by a proprietary algorithm that was developed by Higgs, et al. and has been previously described in detail. (See Higgs, R. E., Knierman, M. D., Gelfanova, V., Butler, J. P., Hale, J. E. (2005) Comprehensive label-free method for the relative quantification of proteins from biological samples, J Proteome Res. 4, 1442-1450; and Higgs, R. E., Knierman, M. D., Freeman, A. B., Gelbert, L. M., Patil, S. T., Hale, J. E. (2007) Estimating the Statistical Significance of Peptide Identifications from Shotgun Proteomics Experiments, J Proteome Res. 4, 1758-1767).

Signatures: Briefly, signatures of proteins were derived obtained using one of several classification model fitting algorithm, with the random-forest or simulated annealing signature derivation method, using machine-learning algorithms for classifying AD versus Control subjects. More specifically, a subset of significant proteins was first filtered out using a robust t-statistic. Signatures were derived using one of the following methods: 1) Relative importance scores from Random Forests algorithm described above, and 2) Simulated Annealing. These derived signatures were then used in one of the following classification algorithms: 1) Linear Discriminant Analysis (LDA), 2) Diagonal Linear Discriminant Analysis (DLDA), 3) Diagonal Quadratic Discriminant Analysis (DQDA), 4) Random Forests, 5) Support Vector Machines, 6) Neural Network, and 7) k-Nearest Neighbor method. Signatures from the above combinations of algorithms were then evaluated for their ability to correctly classify AD and Control subjects using 10 iterations of fully-embedded 5-fold stratified cross-validation. Out of the numerous algorithms and signatures evaluated as described above, the best performing signatures were reported. Substantially the same procedure was carried out for the data from peptides to derive optimal peptide signatures for classifying AD and Control subjects.

Information on the subjects is shown in Table 1. The donors shown in black all were diagnosed with Alzheimer's disease. The MMSE, age and sex of the donors is shown. The donors shown in red are age-matched controls.

TABLE 1

| SUBJECT | | | | MMSE | MMSE | SUBJECT | | | |
|---|---|---|---|---|---|---|---|---|---|
| ID # | AGE | GENDER | DIAGNOSIS | VISIT 1 | VISIT 2 | ID # | AGE | GENDER | DIAGNOSIS |
| 8001 | 83 | M | AD | 17 | 13 | 7856 | 72 | M | Control |
| 8005 | 80 | M | AD | 17 | 20 | 7857 | 73 | M | Control |
| 8006 | 91 | M | AD | 22 | 25 | 7858 | 76 | M | Control |
| 8056 | 75 | M | AD | 15 | 17 | 7860 | 77 | M | Control |
| 8058 | 72 | F | AD | 15 | 11 | 7848 | 80 | M | Control |
| 8026 | 78 | F | AD | 14 | | 7810 | 81 | M | Control |
| 8037 | 78 | F | AD | 14 | | 7815 | 84 | F | Control |
| 8059 | 79 | F | AD | 14 | | 7816 | 85 | F | Control |
| 8038 | 76 | M | AD | 15 | | 7841 | 89 | F | Control |
| 8007 | 78 | F | AD | 16 | | 7811 | 84 | F | Control |
| 8060 | 79 | M | AD | 16 | | | | | |
| 8061 | 82 | F | AD | 16 | | | | | |
| 8064 | 70 | M | AD | 16 | | | | | |
| 8050 | 79 | M | AD | 17 | | | | | |
| 8040 | 87 | M | AD | 19 | | | | | |

892 proteins and 4072 peptides were identified in the CSF. Log transformed quantile-normalized AUC values for each protein and peptide were used for all the data analysis.

Univariate Analysis: The objective of the univariate analysis was to analyze each protein and each peptide one at a time in order to identify those that have significantly different expression between AD and Control groups.

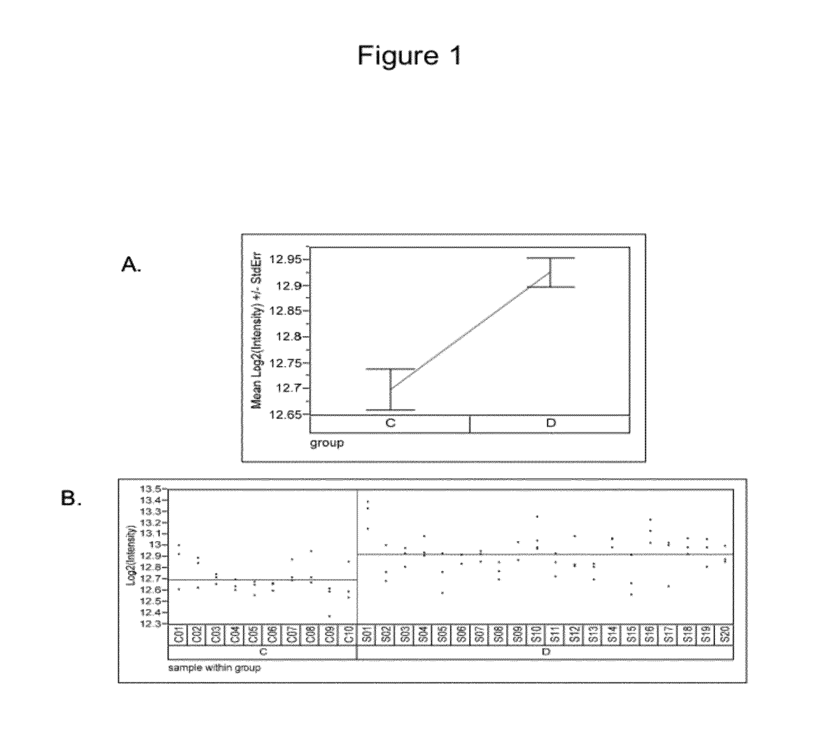

The significance of each protein was assessed via analysis of covariance (ANCOVA) after adjusting for any age and gender differences, and was expressed in terms of the false positive rate (p-value). Seventy three (73) proteins were statistically significant at p<0.05 threshold; these are reported in Table 2A, along with the corresponding volcanic fold change (VFC), % coefficient of variation (CV) and p-value. Positive value of VFC represents an elevation in AD relative to Control and negative value represents an elevation in Control relative to AD by the indicated value. An example of a protein with 1.16 fold higher expression in AD relative to Control (isoform A protein (Protein ID: IPI00001364.2) is shown in FIG. 1. The % CV values represent the total variation in the proteins measured (inter-subject variation plus the technical/analytical variation).

In addition, a more stringent permutation-based nonparametric test using the Significant Analysis of Microarrays (SAM) approach (see Tusher, Tibshirani and Chu, 2001, “Significance analysis of microarrays applied to the ionizing radiation response” PNAS 98: 5116-5121) was used to determine the false discovery rate (q-value) of the significant proteins. This approach accounts for the multiplicity issues that arise due to the simultaneous evaluation of the significance of several proteins. Those proteins that had q<0.1 were reported as being statistically significant under this more rigorous criterion. Out of the 73 proteins in Table 2A that are statistically significant at p<0.05, the first 16 proteins met this more stringent criteria of q<0.1. The rest of the proteins that had q>0.1 are italicized. These top 16 proteins can be considered as a more robust list of proteins with significantly different expression levels between the AD patients and age matched Control subjects.

Similar to the analysis carried out for each protein, each of the 4072 peptides was then analyzed one at a time using the same statistical methods described above. 108 peptides corresponding to 36 proteins were statistically significant at p<0.5 out of which 64 peptides corresponding to 24 proteins were significant under the more stringent false discovery rate (q-value) of q<0.1. These 108 peptides are listed in Table 2B with the peptide ID, corresponding protein ID, protein annotation, peptide sequence, volcanic fold change, % coefficient of variation, p-value and q-value. Those that did not meet the stringent q<0.1 criteria are italicized.

TABLE 2A

| # | Protein_ID | Protein Annotation | VFC | % CV | p-value | q-value |
|---|---|---|---|---|---|---|
| 1 | IPI00001364.2 | _Isoform_A_of_GC-rich_sequence_DNA-binding_factor_homolog | 1.16 | 8.99 | 0.0006 | <0.05 |
| 2 | IPI00006046.4 | _Zinc_finger_protein_536 | 1.2 | 11.87 | 0.0008 | <0.05 |
| 3 | IPI00023019.1 | _Isoform_1_of_Sex_hormone-binding_globulin | −1.22 | 14.09 | 0.0024 | 0.053 |
| 4 | IPI00001510.1 | _Isoform_1_of_Protocadherin_alpha-13 | −1.17 | 11.79 | 0.0031 | 0.053 |
| 5 | IPI00293887.4 | _Isoform_2_of_StAR-related_lipid_transfer_protein_8 | −1.11 | 7.71 | 0.0043 | 0.053 |
| 6 | IPI00032423.2 | _Probable_ATP-dependent_RNA_helicase_DDX52 | −1.29 | 20.49 | 0.0052 | 0.053 |
| 7 | IPI00848198.1 | _Conserved_hypothetical_protein | −1.17 | 12.47 | 0.0060 | 0.053 |
| 8 | IPI00328762.5 | _Isoform_1_of_ATP-binding_cassette_sub-family_A_member_13 | −1.11 | 8.73 | 0.0063 | 0.053 |
| 9 | IPI00164012.1 | _Actin-like_protein_7A | −1.14 | 11.3 | 0.0098 | 0.053 |
| 10 | IPI00219018.7 | _Glyceraldehyde-3-phosphate_dehydrogenase | −1.1 | 8.29 | 0.0110 | 0.053 |
| 11 | IPI00004671.2 | _Golgin_subfamily_B_member_1 | −1.25 | 20.6 | 0.0139 | 0.053 |
| 12 | IPI00031410.1 | _FKBP12-rapamycin_complex-associated_protein | −1.11 | 9.76 | 0.0141 | 0.053 |
| 13 | IPI00645561.2 | _G-protein_coupled_receptor_112 | −1.14 | 13.19 | 0.0197 | 0.053 |
| 14 | IPI00418340.6 | _Isoform_1_of_GC-rich_sequence_DNA-binding_factor | −1.13 | 13.9 | 0.0375 | 0.053 |
| 15 | IPI00807602.1 | _Serine/threonine-protein_kinase_ULK4 | 1.2 | 13.46 | 0.0025 | 0.099 |
| 16 | IPI00059975.3 | _Isoform_2_of_Synaptotagmin-like_protein_2 | 1.23 | 19.1 | 0.0132 | 0.099 |
| 17 | IPI00018747.2 | _Isoform_1_of_Tripartite_motif-containing_protein_45 | −1.51 | 34 | 0.0060 | q > 0.1 |
| 18 | IPI00103604.2 | _Voltage-dependent_calcium_channel_gamma-8_subunit | 1.26 | 18.88 | 0.0066 | q > 0.1 |
| 19 | IPI00011218.1 | _Macrophage_colony-stimulating_factor_1_receptor | 1.17 | 14.19 | 0.0123 | q > 0.1 |
| 20 | IPI00013945.1 | _Isoform_1_of_Uromodulin | −1.26 | 21.45 | 0.0128 | q > 0.1 |
| 21 | IPI00027721.1 | _Isoform_1_of_Alpha-type_platelet-derived_growth_factor_receptor | 1.83 | 59.93 | 0.0138 | q > 0.1 |
| 22 | IPI00015117.2 | _Isoform_Long_of_Laminin_subunit_gamma-2 | 1.78 | 56.85 | 0.0139 | q > 0.1 |
| 23 | IPI00298393.3 | _cDNA_FLJ38738_fis, _clone_KIDNE2011508, _highly_similar_to_{—} | −1.24 | 20.02 | 0.0140 | q > 0.1 |
| | | Homo_sapiens_hNBL4 | | | | |
| 24 | IPI00793423.1 | _14_kDa_protein | −1.14 | 12.19 | 0.0142 | q > 0.1 |
| 25 | IPI00335009.11 | _similar_to_hemicentin_2 | −1.19 | 16.38 | 0.0144 | q > 0.1 |
| 26 | IPI00032958.3 | _Isoform_2_of_Actin-binding_protein_anillin | −1.36 | 29.24 | 0.0145 | q > 0.1 |
| 27 | IPI00044683.1 | _Isoform_1_of_Amyotrophic_lateral_sclerosis_2_chromosomal_{—} | 1.77 | 56.97 | 0.0147 | q > 0.1 |
| | | region_candidate_gene_4_protein | | | | |
| 28 | IPI00294216.3 | _Delta-sarcoglycan | −1.23 | 19.32 | 0.0154 | q > 0.1 |
| 29 | IPI00029061.3 | _Selenoprotein_P | −1.09 | 8.49 | 0.0157 | q > 0.1 |
| 30 | IPI00658050.1 | _CD225_family_protein_FLJ76511 | −1.19 | 16.33 | 0.0161 | q > 0.1 |
| 31 | IPI00103994.4 | _Leucyl-tRNA_synthetase, _cytoplasmic | −1.17 | 15.69 | 0.0194 | q > 0.1 |
| 32 | IPI00166945.3 | _Protein_FAM101B | −1.34 | 29.48 | 0.0194 | q > 0.1 |
| 33 | IPI00019449.1 | DQB1; HLA-DRB4; HLA-DRB2; HLA-DQB2; | −1.22 | 19.36 | 0.0201 | q > 0.1 |
| | | hCG_1998957; LOC100133484; | | | | |
| | | ZNF749; HLA-DRB5; HLA-DRB1; RNASE2; | | | | |
| | | HLA-DRB3; LOC100133661; | | | | |
| | | LOC100133583_Non-secretory_ribonuclease | | | | |
| 34 | IPI00044369.2 | _Isoform_1_of_Plexin_domain-containing_protein_2 | −1.17 | 15.11 | 0.0203 | q > 0.1 |
| 35 | IPI100000828.3 | _Proenkephalin_A | −1.15 | 14.17 | 0.0203 | q > 0.1 |
| 36 | IPI00152311.4 | _Isoform_1_of_Uncharacterized_protein_C3orf38 | 1.63 | 51.05 | 0.0204 | q > 0.1 |
| 37 | IPI00029468.1 | _Alpha-centractin | −1.34 | 29.92 | 0.0213 | q > 0.1 |
| 38 | IPI00071929.5 | _cDNA_FLJ77573 | −1.31 | 27.7 | 0.0241 | q > 0.1 |
| 39 | IPI00410600.3 | _Isoform_3_of_Voltage-dependent_calcium_channel_subunit_alpha- | −1.27 | 24.46 | 0.0241 | q > 0.1 |
| | | 2/delta-2 | | | | |
| 40 | IPI00472200.4 | _Isoform_B_of_Collagen_alpha-6(IV)_chain | −1.1 | 10.05 | 0.0278 | q > 0.1 |
| 41 | IPI00015864.1 | _2-5A-dependent_ribonuclease | −1.48 | 42.98 | 0.0292 | q > 0.1 |
| 42 | IPI00010732.1 | _Parathyroid_hormone/parathyroid_hormone- | −1.53 | 47.21 | 0.0295 | q > 0.1 |
| | | related_peptide_receptor | | | | |
| 43 | IPI00887377.1 | _similar_to_rCG63049 | −1.09 | 9.45 | 0.0299 | q > 0.1 |
| 44 | IPI00011264.1 | _Complement_factor_H-related_protein_1 | −1.09 | 9.46 | 0.0304 | q > 0.1 |
| 45 | IPI00006608.1 | _Isoform_APP770_of_Amyloid_beta_A4_protein_(Fragment) | −1.16 | 15.55 | 0.0318 | q > 0.1 |
| 46 | IPI00329028.2 | _WD_repeat-containing_protein_5B | −1.17 | 16.95 | 0.0324 | q > 0.1 |
| 47 | IPI00853062.1 | _Uncharacterized_protein_RPS9 | 1.35 | 33.76 | 0.0332 | q > 0.1 |
| 48 | IPI00398505.5 | _ubiquitin_specific_protease_24 | −1.34 | 32.65 | 0.0345 | q > 0.1 |
| 49 | IPI00872163.1 | _Similar_to_ATPase, _Ca++_transporting, _cardiac_muscle, _fast_{—} | −1.25 | 24.8 | 0.0347 | q > 0.1 |
| | | twitch_1_(Fragment) | | | | |
| 50 | IPI00008603.1 | _Actin,_aortic_smooth_muscle | 1.07 | 7.28 | 0.0353 | q > 0.1 |
| 51 | IPI00220656.4 | _T-complex_protein_1_subunit_zeta-2 | −1.2 | 20.04 | 0.0361 | q > 0.1 |
| 52 | IPI00016988.20 | _cDNA, _FLJ95601, _highly_similar_to_Homo_sapiens_WD_repeat_{—} | −1.2 | 20.04 | 0.0370 | q > 0.1 |
| | | domain_13_(WDR13), _mRNA | | | | |
| 53 | IPI00478916.4 | _DNA_cross-link_repair_1A_protein | −1.25 | 24.97 | 0.0377 | q > 0.1 |
| 54 | IPI00032258.4 | _Complement_C4-A | 1.14 | 14.96 | 0.0382 | q > 0.1 |
| 55 | IPI00254408.6 | _bromodomain_PHD_finger_transcription_factor_isoform_1 | 1.25 | 25.42 | 0.0384 | q > 0.1 |
| 56 | IPI00022463.1 | _Serotransferrin | −1.06 | 6.16 | 0.0390 | q > 0.1 |
| 57 | IPI00157790.7 | _KIAA0368_protein | 2.02 | 93.18 | 0.0398 | q > 0.1 |
| 58 | IPI00017921.7 | _Isoform_2_of_Protein_bicaudal_C_homolog_1 | −1.24 | 24.96 | 0.0406 | q > 0.1 |
| 59 | IPI00293963.4 | _Isoform_1_of_Chromodomain_Y-like_protein | 1.47 | 45.87 | 0.0406 | q > 0.1 |
| 60 | IPI00063800.1 | _Zinc_finger_protein_496 | 1.46 | 45.37 | 0.0420 | q > 0.1 |
| 61 | IPI00247295.3 | _Isoform_4_of_Nesprin-1 | −1.24 | 24.93 | 0.0422 | q > 0.1 |
| 62 | IPI00217346.2 | _zinc_finger_protein, _multitype_1 | −1.08 | 8.82 | 0.0429 | q > 0.1 |
| 63 | IPI00020088.1 | _Interleukin-26 | −1.18 | 19.12 | 0.0431 | q > 0.1 |
| 64 | IPI00019209.1 | _Semaphorin-3C | 1.16 | 17.16 | 0.0431 | q > 0.1 |
| 65 | IPI00739099.2 | _Collagen_alpha-2(V)_chain | −1.15 | 16.32 | 0.0433 | q > 0.1 |
| 66 | IPI00215983.3 | _Carbonic_anhydrase_1 | 1.35 | 35.49 | 0.0446 | q > 0.1 |
| 67 | IPI00418163.3 | _complement_component_4B_preproprotein | 1.14 | 15.17 | 0.0449 | q > 0.1 |
| 68 | IPI00297284.1 | _Insulin-like_growth_factor-binding_protein_2 | 1.09 | 10.2 | 0.0457 | q > 0.1 |
| 69 | IPI00010172.1 | _Isoform_Short_of_Gastric_inhibitory_polypeptide_receptor | 1.1 | 10.84 | 0.0470 | q > 0.1 |
| 70 | IPI00025880.2 | _Myosin-7 | −1.31 | 32.8 | 0.0479 | q > 0.1 |
| 71 | IPI00218052.5 | _Isoform_1_of_WD_repeat_and_FYVE_domain- | −1.08 | 9.46 | 0.0491 | q > 0.1 |
| | | containing_protein_3 | | | | |
| 72 | IPI00016095.1 | _Transcription_termination_factor, _mitochondrial | −1.26 | 27.52 | 0.0493 | q > 0.1 |
| 73 | IPI00060181.1 | _EF-hand_domain-containing_protein_D2 | −1.17 | 18.48 | 0.0500 | q > 0.1 |

TABLE 2B

| SEQ | | | | | | | | |
|---|---|---|---|---|---|---|---|---|
| ID | | | Peptide | | | | p- | |
| NO: | Protein ID | Protein Annotation | ID | Sequence | VFC | % CV | value | q-value |
| 1 | IPI00032258.4 | _Complement_C4-A | 442 | TTNIQGINLLFSSR | 1.48 | 25.25 | 0.0007 | 0 |
| 2 | | | 1699 | ILTVPGHLDEMQLDI | 1.21 | 13.3 | 0.0018 | 0.048 |
| | | | | QAR | | | | |
| 3 | | | 2518 | ASAGLLGAHAAAITA | 1.15 | 9.55 | 0.0018 | 0.048 |
| | | | | Y | | | | |
| 4 | | | 615 | VTASDPLDTLGSEGA | 1.25 | 18.64 | 0.0071 | 0.048 |
| | | | | LSPGGVASLLR | | | | |
| 5 | | | 1626 | VGLSGMAIADVTLLS | 1.19 | 16.18 | 0.0164 | 0.056 |
| | | | | GFHALR | | | | |
| 6 | | | 1665 | DDPDAPLQPVTPLQL | 1.21 | 13.44 | 0.0019 | 0.056 |
| | | | | FEGR | | | | |
| 7 | | | 2232 | ITPGKPYILTVPGHL | 1.22 | 13.86 | 0.0017 | 0.056 |
| | | | | DEMQLDIQAR | | | | |
| 8 | | | 2422 | LLLFSPSVVHLGVPL | 1.23 | 19.07 | 0.0137 | 0.056 |
| | | | | SVGVQLQDVPR | | | | |
| 9 | | | 264 | AEFQDALEK | 1.19 | 17.24 | 0.0216 | 0.056 |
| 10 | | | 2889 | SCGLHQLLR | 1.23 | 21.09 | 0.0239 | 0.056 |
| 11 | | | 459 | LNMGITDLQGLR | 1.27 | 29.44 | 0.0517 | 0.056 |
| 12 | | | 731 | VTASDPLDTLGSEAG | 1.23 | 24.64 | 0.0510 | 0.056 |
| | | | | LSPGGVASLLR | | | | |
| 13 | | | 1629 | LELSVDGAK | 1.17 | 14.6 | 0.0157 | 0.089 |
| 14 | | | 1922 | LQETSNWLLSQQQA | 1.72 | 49.01 | 0.0086 | 0.089 |
| | | | | DGSFQDPCPVLDR | | | | |
| 15 | | | 366 | LLLFSPSVVHLGVPL | 1.3 | 28.86 | 0.0312 | 0.089 |
| | | | | SVGVQLQDVPR | | | | |
| 16 | | | 911 | FGLLDEDGKK | 1.2 | 22.59 | 0.0563 | 0.089 |
| 17 | | | 926 | HLVPGAPFLLQALVR | 1.19 | 18.34 | 0.0257 | 0.089 |
| 18 | | | 238 | DFALLSLQVPLK | 1.22 | 22.25 | 0.0392 | q > 0.1 |
| 19 | | | 2858 | EELVYELNPLDHR | 1.19 | 15.9 | 0.0117 | q > 0.1 |
| 20 | | | 371 | FQILTLWLPDSLTTW | 1.26 | 27.76 | 0.048 | q > 0.1 |
| | | | | EIHGLSLSK | | | | |
| 21 | | | 389 | VTASDPLDTLGSEGA | 1.27 | 31.98 | 0.0714 | q > 0.1 |
| | | | | LSPGGVASLLR | | | | |
| 22 | | | 519 | ALEILQEEDLIDEDD | 1.26 | 24.81 | 0.0309 | q > 0.1 |
| | | | | IPVR | | | | |
| 23 | | | 162 | VDVQAGACEGK | 1.2 | 22.04 | 0.0477 | q > 0.1 |
| 24 | | | 532 | DHAVDLIQK | 1.21 | 26.32 | 0.0881 | q > 0.1 |
| 25 | IPI00418163.3 | _complement_component_4B_ | 442 | TTNIQGINLLFSSR | 1.48 | 25.5 | 0.0007 | 0 |
| 26 | | preproprotein | 1699 | ILTVPGHLDEMQLDI | 1.21 | 13.3 | 0.001 | 0.048 |
| | | | | QAR | | | | |
| 27 | | | 2518 | ASAGLLGAHAAAITA | 1.15 | 9.55 | 0.0018 | 0.048 |
| | | | | Y | | | | |
| 28 | | | 615 | VTASDPLDTLGSEGA | 1.25 | 18.64 | 0.0071 | 0.048 |
| | | | | LSPGGVASLLR | | | | |
| 29 | | | 1626 | VGLSGMAIADVTLLS | 1.19 | 16.18 | 0.0164 | 0.056 |
| | | | | GFHALR | | | | |
| 30 | | | 1665 | DDPDAPLQPVTPLQL | 1.21 | 13.44 | 0.0019 | 0.056 |
| | | | | FEGR | | | | |
| 31 | | | 2232 | ITPGKPYILTVPGHL | 1.22 | 13.86 | 0.0017 | 0.056 |
| | | | | DEMQLDIQAR | | | | |
| 32 | | | 2422 | LLLFSPSVVHLGVPL | 1.23 | 19.07 | 0.0137 | 0.056 |
| | | | | SVGVQLQDVPR | | | | |
| 33 | | | 264 | AEFQDALEK | 1.19 | 17.24 | 0.0216 | 0.056 |
| 34 | | | 2889 | SCGLHQLLR | 1.23 | 21.09 | 0.0239 | 0.056 |
| 35 | | | 459 | LNMGITDLQGLR | 1.27 | 29.44 | 0.0517 | 0.056 |
| 36 | | | 731 | VTASDPLDTLGSEGA | 1.23 | 24.64 | 0.0510 | 0.056 |
| | | | | LSPGGVASLLR | | | | |
| 37 | | | 1076 | AEMADQAAAWLTR | 1.26 | 19.09 | 0.0064 | 0.089 |
| 38 | | | 1629 | LELSVDGAK | 1.17 | 14.6 | 0.0157 | 0.089 |
| 39 | | | 366 | LLLFSPSVVHLGVPL | 1.3 | 28.86 | 0.0312 | 0.089 |
| | | | | SVGVQLQDVPR | | | | |
| 40 | | | 911 | FGLLDEDGKK | 1.2 | 22.59 | 0.0563 | 0.089 |
| 41 | | | 926 | HLVPGAPFLLQALVR | 1.19 | 18.34 | 0.0257 | 0.089 |
| 42 | | | 238 | DFALLSLQVPLK | 1.22 | 22.25 | 0.0392 | q > 0.1 |
| 43 | | | 2858 | EELVYELNPLDHR | 1.19 | 15.97 | 0.0117 | q > 0.1 |
| 44 | | | 371 | FQILTLWLPDSLTTW | 1.26 | 27.67 | 0.0487 | q > 0.1 |
| | | | | EIHGLSLSK | | | | |
| 45 | | | 389 | VTASDPLDTLGSEGA | 1.27 | 31.98 | 0.0714 | q > 0.1 |
| | | | | LSPGGVASLLR | | | | |
| 46 | | | 519 | ALEILQEEDLIDEDD | 1.26 | 24.81 | 0.0309 | q > 0.1 |
| | | | | IPVR | | | | |
| 47 | | | 162 | VDVQAGACEGK | 1.2 | 22.04 | 0.047 | q > 0.1 |
| 48 | | | 532 | DHAVDLIQK | 1.21 | 26.32 | 0.0881 | q > 0.1 |
| 49 | IPI00550991.3 | _cDNA_FLJ35730_fis,_clone_ | 2591 | EIGELYLPK | 1.16 | 9.06 | 0.0006 | 0 |
| 50 | | TESTI2003131, _highly_similar_ | 597 | DEELSCTVVELK | 1.19 | 13.55 | 0.0042 | 0.056 |
| 51 | | to_ALPHA-1-ANTICHYMOTRYPSIN | 769 | HPNSPLDEENLTQE | 1.23 | 17.46 | 0.0080 | 0.056 |
| | | | | NQDR | | | | |
| 52 | | | 1961 | LYGSEAFATDFQDS | 1.13 | 12.67 | 0.0260 | 0.089 |
| | | | | AAAK | | | | |
| 53 | | | 150 | HPNSPLDEENLTQE | 1.21 | 17.93 | 0.0166 | q > 0.1 |
| | | | | NQDR | | | | |
| 54 | | | 1592 | ITLLSALVETR | 1.18 | 18.81 | 0.0403 | q > 0.1 |
| 55 | | | 1723 | AVLDVFEEGTEASAA | 1.18 | 15.65 | 0.0155 | q > 0.1 |
| | | | | TAVK | | | | |
| 56 | | | 2303 | ADLSGITGAR | 1.18 | 17.24 | 0.0234 | q > 0.1 |
| 57 | | | 1379 | AVLDVFEEGTEASAA | 1.13 | 12.54 | 0.0264 | q > 0.1 |
| | | | | TAVK | | | | |
| 58 | | | 2727 | GTHVDLGLASANVDF | 1.11 | 9.58 | 0.0122 | q > 0.1 |
| | | | | AF | | | | |
| 59 | | | 302 | EIGELYLPK | 1.15 | 13.81 | 0.0241 | q > 0.1 |
| 60 | IPI00654755.3 | _Hemoglobin_subunit_beta | 2920 | VVAGVANALAHK | 1.61 | 35.28 | 0.0024 | 0.056 |
| 61 | | | 1202 | LLVVYPWTQR | 1.44 | 39.72 | 0.0299 | q > 0.1 |
| 62 | | | 1825 | FFESFGDLSTPDAV | 1.75 | 56.35 | 0.0152 | q > 0.1 |
| | | | | MGNPK | | | | |
| 63 | | | 1852 | CVLAHHFGK | 1.71 | 69.14 | 0.0457 | q > 0.1 |
| 64 | IPI00783987.2 | _Complement_C3_(Fragment) | 1073 | QKPDGVFQEDAPVI | 1.18 | 12.46 | 0.0038 | 0.056 |
| | | | | HQEMIGGLR | | | | |
| 65 | | | 1505 | IPIEDGSGEVVLSR | 1.14 | 1.054 | 0.0049 | 0.089 |
| 66 | | | 230 | SSLSVPYVIVPLK | 1.18 | 13.5 | 0.0062 | 0.089 |
| 67 | | | 1921 | GQDLVVLPLSITTDF | 1.14 | 13.58 | 0.0255 | q > 0.1 |
| | | | | IPSFR | | | | |
| 68 | IPI00887739.1 | _hypothetical_protein, _ | 1073 | QKPDGVFQEDAPVIH | 1.18 | 12.46 | 0.0038 | 0.056 |
| | | partial | | QEMIGGLR | | | | |
| 69 | | | 1505 | IPIEDGSGEVVLSR | 1.14 | 10.54 | 0.0049 | 0.089 |
| 66 | | | 230 | SSLSVPYVIVPLK | 1.18 | 13.5 | 0.0062 | 0.089 |
| 71 | | | 1921 | GQDLVVLPLSITTDF | 1.14 | 13.58 | 0.0255 | q > 0.1 |
| | | | | IPSFR | | | | |
| 72 | IPI00022463.1 | _Serotransferrin | 1173 | KPVDEYKDCHLAQV | −1.16 | 8.83 | 0.0004 | 0 |
| | | | | PSHTVVAR | | | | |
| 73 | | | 16 | EGYYGYTGAFR | −1.27 | 32.44 | 0.0791 | 0 |
| 74 | | | 3331 | SDNCEDTPEAGYF | 1.15 | 12.32 | 0.0122 | q > 0.1 |
| 75 | IPI00215983.3 | _Carbonic_anhydrase_1 | 2258 | HDTSLKPISVSYNPA | 1.42 | 34.21 | 0.0168 | q > 0.1 |
| | | | | TAK | | | | |
| 76 | | | 2542 | ADGLAVIGVLMK | 1.52 | 43.68 | 0.0217 | q > 0.1 |
| 77 | | | 4229 | LYPIANGNNQSPVDI | 1.66 | 56.65 | 0.0270 | q > 0.1 |
| | | | | K | | | | |
| 78 | IPI00473011.3 | _Hemoglobin_subunit_delta | 2920 | VVAGVANALAHK | 1.61 | 35.28 | 0.0024 | 0.056 |
| 79 | | | 1202 | LLVVYPWTQR | 1.44 | 39.72 | 0.029 | q > 0.1 |
| 80 | IPI00001364.2 | _Isoform A_of GC-rich_sequence_ | 3990 | LEGSSGGIGER | 1.16 | 8.99 | 0.0006 | 0 |
| | | DNA-binding_factor_homolog | | | | | | |
| 81 | IPI00006046.4 | _Zinc_finger_protein_536 | 2550 | GNLKIHLR | 1.2 | 11.87 | 0.0008 | 0 |
| 82 | IPI00021841.1 | _Apolipoprotein_A-I | 1616 | AHVDALR | −1.18 | 8.09 | 0.0000 | 0 |
| 83 | IPI00032292.1 | _Metalloproteinase_inhibitor_1 | 1998 | LQDGLLHITTCSFVA | 1.28 | 19.36 | 0.0044 | 0 |
| | | | | PWNSLSLAQR | | | | |
| 84 | IPI00410714.5 | _Hemoglobin_subunit_alpha | 1952 | KVADALTNAVAHVD | 1.5 | 25.64 | 0.0007 | 0 |
| | | | | DMPNALSALSDLHA | | | | |
| | | | | HK | | | | |
| 85 | IPI00807602.1 | Serineithreonine-protein_ | 3412 | ILCEDPLPPIPKDSS | 1.2 | 13.46 | 0.0025 | 0.048 |
| | | kinase_ULK4 | | RPK | | | | |
| 86 | IPI00059975.3 | _Isoform_2_of_Synaptotagmin- | 3749 | PSSLTNLSSSSGMTS | 1.23 | 19.1 | 0.0132 | 0.056 |
| | | like_protein_2 | | LSSVSGSVMSV | | | | |
| 87 | IPI00418194.3 | Breast_cancer-associated_ | 3749 | PSSLTNLSSSSGMTS | 1.23 | 19.1 | 0.0132 | 0.056 |
| | | antigen_SGA-_72M | | LSSVSGSVMSV | | | | |
| 88 | IPI00478003.1 | _Alpha-2-macroglobulin | 1676 | SSSNEEVMFLTVQV | 1.14 | 9.88 | 0.0031 | 0.056 |
| | | | | K | | | | |
| 89 | IPI00009028.1 | _Tetranectin | 3214 | GGTLSTPQTGSEND | 1.29 | 22.77 | 0.0118 | 0.089 |
| 90 | IPI00015117.2 | Isoform_Long_of_Laminin_ | 3162 | CLPGFHMLTDAGCT | 1.78 | 56.85 | 0.0139 | 0.089 |
| | | subunit_gamma-2 | | gamma-2 | | | | |
| 91 | IPI00016915.1 | Insulin-like_growth_factor- | 3346 | ITVVDALHEIPVK | 1.18 | 15.95 | 0.0170 | 0.089 |
| | | binding_protein_7 | | protein_7 | | | | |
| 92 | IPI00044683.1 | Isoform_1_of_ | 3839 | NENGIDAEPAEEAVI | 1.77 | 56.97 | 0.0147 | 0.089 |
| | | Amyotrophiciateral_sclerosis_ | | QKPR | | | | |
| | | 2_chromosomal_region_ | | | | | | |
| | | candidate_gene_4_protein | | | | | | |
| 93 | IPI00063800.1 | _Zinc_finger_protein_496 | 3286 | PESGEQAVAAVEAL | 1.46 | 45.37 | 0.0420 | 0.089 |
| | | | | ER | | | | |
| 94 | IPI00103604.2 | Voltage- | 3602 | AFGGAAGGAGGGG | 1.26 | 18.88 | 0.0066 | 0.089 |
| | | dependent_calcium_channel_ | | GGGGGAGA | | | | |
| | | gamma-8_subunit | | | | | | |
| 95 | IPI00293963.4 | _Isoform_1_of_Chromodomain_Y- | 3328 | CNMKMELEQANER | 1.47 | 45.87 | 0.0406 | 0.089 |
| | | like_protein | | | | | | |
| 96 | 126608 | LYSOZYME_Spiked_Standard_(HEN) | 2376 | KIVSDGNGMNAWVA | 1.11 | 9.41 | 0.0155 | q > 0.1 |
| | | | | WR | | | | |
| 97 | IPI00024046.1 | _Cadherin-13 | 1992 | EDLDCTPGFQQK | 1.14 | 12.65 | 0.0185 | q > 0.1 |
| 98 | IPI00027721.1 | _Isoform_1_of Alpha-type_ | 3193 | QADTTQYVPMLER | 1.83 | 59.93 | 0.0138 | q > 0.1 |
| | | platelet-derived_growth_ | | | | | | |
| | | factor_receptor | | | | | | |
| 99 | IPI00217471.3 | _Hemoglobin_subunit_epsilon | 1202 | LLVVYPWTQR | 1.44 | 39.72 | 0.0299 | q > 0.1 |
| 100 | IPI00294004.1 | _Vitamin_K-dependent_protein_S | 1422 | ITTGGDVINNGLWNM | 1.08 | 8.29 | 0.0317 | q > 0.1 |
| | | | | VSVEELEHSISIK | | | | |
| 101 | IPI00886899.1 | _similar_to_hCG1646049 | 3421 | SSGQAGNKSER | 1.19 | 21.65 | 0.0575 | q > 0.1 |
| 102 | IPI00022283.1 | _Trefoil factor _1 | 3080 | MATMENK | 1.75 | 58.13 | 0.0180 | q > 0.1 |
| 103 | IPI00013945.1 | _Isoform_1_of_Uromodulin | 3006 | FVGQGGAR | −1.53 | 30.45 | 0.0019 | q > 0.1 |
| 104 | IPI00152311.4 | _Isoform_1_of_Uncharacterized_ | 3712 | FINLKIMGESSLAPG | 1.63 | 51.05 | 0.0204 | q > 0.1 |
| | | protein_C3orf38 | | TLPKPSVK | | | | |
| 105 | IPI00291262.3 | _Clusterin | 2219 | TLLSNLEEAKK | −1.24 | 14.66 | 0.0015 | q > 0.1 |
| 106 | IPI00006601.5 | _Secretogranin-1 | 1395 | GYPGVQAPEDLEWE | −3.07 | 108.08 | 0.0048 | q > 0.1 |
| | | | | R | | | | |
| 107 | | | 2295 | GEDSSEEKHLEEPG | −1.73 | 42.46 | 0.0032 | q > 0.1 |
| | | | | ETQNAFLNER | 1.73 | 6 | 2 | |
| 106 | | | 2406 | GYPGVQAPEDLEWE | 2.66 | 129.96 | 0.0245 | q > 0.1 |
| | | | | R | | | | |

TABLE 2C

| # | Protein ID | Protein Annotation |
|---|---|---|
| 1 | 126608 | LYSOZYME_Spiked_Standard_(HEN) |
| 2 | IPI00000828.3 | _Proenkephalin_A |
| 3 | IPI00001364.2 | _Isoform_A_of_GC-rich_sequence_DNA-binding_factor_homolog |
| 4 | IPI00001510.1 | _Isoform_1_of_Protocadherin_alpha-13 |
| 5 | IPI00004671.2 | _Golgin_subfamily_B_member_1 |
| 6 | IPI00006046.4 | _Zinc_finger_protein_536 |
| 7 | IPI00006601.5 | _Secretogranin-1 |
| 8 | IPI00006608.1 | _Isoform_APP770_of_Amyloid_beta_A4_protein_(Fragment) |
| 9 | IPI00008603.1 | _Actin,_aortic_smooth_muscle |
| 10 | IPI00009028.1 | _Tetranectin |
| 11 | IPI00010172.1 | _Isoform_Short_of_Gastric_inhibitory_polypeptide_receptor |
| 12 | IPI00010732.1 | _Parathyroid_hormone/parathyroid_hormone-related_peptide_receptor |
| 13 | IPI00011218.1 | _Macrophage_colony-stimulating_factor_1_receptor |
| 14 | IPI00011264.1 | _Complement_factor_H-related_protein_1 |
| 15 | IPI00013945.1 | _Isoform_1_of_Uromodulin |
| 16 | IPI00015117.2 | _Isoform_Long_of_Laminin_subunit_gamma-2 |
| 17 | IPI00015864.1 | _2-5A-dependent_ribonuclease |
| 18 | IPI00016095.1 | _Transcription_termination_factor,_mitochondrial |
| 19 | IPI00016915.1 | _Insulin-like_growth_factor-binding_protein_7 |
| 20 | IPI00016988.20 | _cDNA,_FLJ95601,_highly_similar_to_Homo_sapiens_WD_repeat_domain_13_(WDR13),_mRNA |
| 21 | IPI00017921.7 | _Isoform_2_of_Protein_bicaudal_C_homolog_1 |
| 22 | IPI00018747.2 | _Isoform_1_of_Tripartite_motif-containing_protein_45 |
| 23 | IPI00019209.1 | _Semaphorin-3C |
| 24 | IPI00019449.1 | DQB1; HLA-DRB4; HLA-DRB2; HLA-DQB2; hCG_1998957; LOC100133484; ZNF749; HLA- |
| | | DRB5; HLA-DRB1; RNASE2; HLA-DRB3; LOC100133661; LOC100133583_Non- |
| | | secretory_ribonuclease |
| 25 | IPI00020088.1 | _Interleukin-26 |
| 26 | IPI00021841.1 | _Apolipoprotein_A-I |
| 27 | IPI00022283.1 | _Trefoil_factor_1 |
| 28 | IPI00022463.1 | _Serotransferrin |
| 29 | IPI00023019.1 | _Isoform_1_of_Sex_hormone-binding_globulin |
| 30 | IPI00024046.1 | _Cadherin-13 |
| 31 | IPI00025880.2 | _Myosin-7 |
| 32 | IPI00027721.1 | _Isoform_1_of_Alpha-type_platelet-derived_growth_factor_receptor |
| 33 | IPI00029061.3 | _Selenoprotein_P |
| 34 | IPI00029468.1 | _Alpha-centractin |
| 35 | IPI00031410.1 | _FKBP12-rapamycin_complex-associated_protein |
| 36 | IPI00032258.4 | _Complement_C4-A |
| 37 | IPI00032292.1 | _Metalloproteinase_inhibitor_1 |
| 38 | IPI00032423.2 | _Probable_ATP-dependent_RNA_helicase_DDX52 |
| 39 | IPI00032958.3 | _Isoform_2_of_Actin-binding_protein_anillin |
| 40 | IPI00044369.2 | _Isoform_1_of_Plexin_domain-containing_protein_2 |
| 41 | IPI00044683.1 | _Isoform_1_of_Amyotrophic_lateral_sclerosis_2_chromosomal_region_candidate_gene_4_protein |
| 42 | IPI00059975.3 | _Isoform_2_of_Synaptotagmin-like_protein_2 |
| 43 | IPI00060181.1 | _EF-hand_domain-containing_protein_D2 |
| 44 | IPI00063800.1 | _Zinc_finger_protein_496 |
| 45 | IPI00071929.5 | _cDNA_FLJ77573 |
| 46 | IPI00103604.2 | _Voltage-dependent_calcium_channel_gamma-8_subunit |
| 47 | IPI00103994.4 | _Leucyl-tRNA_synthetase,_cytoplasmic |
| 48 | IPI00152311.4 | _Isoform_1_of_Uncharacterized_protein_C3orf38 |
| 49 | IPI00157790.7 | _KIAA0368_protein |
| 50 | IPI00164012.1 | _Actin-like_protein_7A |
| 51 | IPI00166945.3 | _Protein_FAM101B |
| 52 | IPI00215983.3 | _Carbonic_anhydrase_1 |
| 53 | IPI00217346.2 | _zinc_finger_protein,_multitype_1 |
| 54 | IPI00217471.3 | _Hemoglobin_subunit_epsilon |
| 55 | IPI00218052.5 | _Isoform_1_of_WD_repeat_and_FYVE_domain-containing_protein_3 |
| 56 | IPI00219018.7 | _Glyceraldehyde-3-phosphate_dehydrogenase |
| 57 | IPI00220656.4 | _T-complex_protein_1_subunit_zeta-2 |
| 58 | IPI00247295.3 | _Isoform_4_of_Nesprin-1 |
| 59 | IPI00254408.6 | _bromodomain_PHD_finger_transcription_factor_isoform_1 |
| 60 | IPI00291262.3 | _Clusterin |
| 61 | IPI00293887.4 | _Isoform_2_of_StAR-related_lipid_transfer_protein_8 |
| 62 | IPI00293963.4 | _Isoform_1_of_Chromodomain_Y-like_protein |
| 63 | IPI00294004.1 | _Vitamin_K-dependent_protein_S |
| 64 | IPI00294216.3 | _Delta-sarcoglycan |
| 65 | IPI00297284.1 | _Insulin-like_growth_factor-binding_protein_2 |
| 66 | IPI00298393.3 | _cDNA_FLJ38738_fis, _clone_KIDNE2011508, _highly_similar_to_Homo_sapiens_hNBL4 |
| 67 | IPI00328762.5 | _Isoform_1_of_ATP-binding_cassette_sub-family_A_member_13 |
| 68 | IPI00329028.2 | _WD_repeat-containing_protein_5B |
| 69 | IPI00335009.11 | _similar_to_hemicentin_2 |
| 70 | IPI00398505.5 | _ubiquitin_specific_protease_24 |
| 71 | IPI00410600.3 | _Isoform_3_of_Voltage-dependent_calcium_channel_subunit_alpha-2/delta-2 |
| 72 | IPI00410714.5 | _Hemoglobin_subunit_alpha |
| 73 | IPI00418163.3 | _complement_component_4B_preproprotein |
| 74 | IPI00418194.3 | _Breast_cancer-associated_antigen_SGA-72M |
| 75 | IPI00418340.6 | _Isoform_1_of_GC-rich_sequence_DNA-binding_factor |
| 76 | IPI00472200.4 | _Isoform_B_of_Collagen_alpha-6(IV)_chain |
| 77 | IPI00473011.3 | _Hemoglobin_subunit_delta |
| 78 | IPI00478003.1 | _Alpha-2-macroglobulin |
| 79 | IPI00478916.4 | _DNA_cross-link_repair_1A_protein |
| 80 | IPI00550991.3 | _cDNA_FLJ35730_fis, _clone_TESTI2003131, _highly_similar_to_ALPHA-1- |
| | | ANTICHYMOTRYPSIN |
| 81 | IPI00645561.2 | _G-protein_coupled_receptor_112 |
| 82 | IPI00654755.3 | _Hemoglobin_subunit_beta |
| 83 | IPI00658050.1 | _CD225_family_protein_FLJ76511 |
| 84 | IPI00739099.2 | _Collagen_alpha-2(V)_chain |
| 85 | IPI00783987.2 | _Complement_C3_(Fragment) |
| 86 | IPI00793423.1 | _14_kDa_protein |
| 87 | IPI00807602.1 | _Serine/threonine-protein_kinase_ULK4 |
| 88 | IPI00848198.1 | _Conserved_hypothetical_protein |
| 89 | IPI00853062.1 | _Uncharacterized_protein_RPS9 |
| 90 | IPI00872163.1 | _Similar_to_ATPase, _Ca++_transporting, _cardiac_muscle, _fast_twitch_1_(Fragment) |
| 91 | IPI00886899.1 | _similar_to_hCG1646049 |
| 92 | IPI00887377.1 | _similar_to_rCG63049 |
| 93 | IPI00887739.1 | _hypothetical_protein, _partial |

Out of the 36 proteins in Table 2B for whom one or more peptides are statistically significant at p<0.05, 16 proteins were also significant in the previous analysis reported in Table 2A. Thus, in addition to the 73 proteins reported as significant at p<0.05 in Table 2A, twenty (20) new proteins are reported as significant at the peptide level in Table 2B. Thus 93 proteins in total have been identified as significant at p<0.05 from these univariate analyses, and these 93 are summarized in the listing of Table 2C, above.

Similarly, out of the 24 proteins in Table 2B for whom one or more peptides are significant at the more stringent criteria of q<0.1, four (4) proteins were also significant in the previous analysis reported in Table 2A. Thus, in addition to the 16 proteins reported as significant at q<0.1 in Table 2A, there are 20 new proteins reported as significant at the peptide level in Table 2B. Thus totally 36 proteins (20+16) have been identified as significant at the more stringent criteria of q<0.1 from these univariate analyses.

Multivariate Analysis: Further analysis of these proteins using machine-learning algorithms provided optimal signatures (composites of proteins) for classifying AD versus Control subjects. A subset of significant proteins was first filtered out using a robust t-statistic. Signatures were derived using one of the following methods: 1) Relative importance scores from Random Forests algorithm (see Breiman, L. (2001), Random Forests, Machine Learning 45(1), 5-32), and 2) Simulated Annealing algorithm (see Cadima, J., Cerdeira, J. Orestes and Minhoto, M. (2004), Computational aspects of algorithms for variable selection in the context of principal components. Computational Statistics & Data Analysis, 47, 225-236). These derived signatures were then used in one of the following classification algorithms: 1) Linear Discriminant Analysis (LDA), 2) Diagonal Linear Discriminant Analysis (DLDA), 3) Diagonal Quadratic Discriminant Analysis (DQDA), 4) Random Forests, 5) Support Vector Machines, 6) Neural Network, and 7) k-Nearest Neighbor method. Signatures from the above combinations of algorithms were then evaluated for their ability to correctly classify AD and Control subjects.

This evaluation was done rigorously using 10 iterations of fully-embedded 5-fold stratified cross-validation. This was carried out by first dividing the original dataset randomly into five equal parts, stratified to ensure that each of these parts had the same balance between AD and Control subjects as was found in the original dataset. Then each part was left out one at a time (test-set), and the remaining four parts were used as a training set to derive the optimal signature and fit the classification model described above; all steps in the analysis procedure (filtering important proteins, deriving signatures, fitting models) were repeated independently for each training set. The models on the training sets were then used to predict the test-sets, and the predictions from all the five test-sets were pooled together to estimate the performance measures, sensitivity (ability to correctly identify AD subjects) and specificity (ability to correctly identify Control subjects). This entire procedure was iterated 10 times to yield Mean and SE (standard error) of sensitivity and specificity.

Out of the numerous algorithms and signatures evaluated as described above, two of the best signatures are summarized in Table 3A below.

TABLE 3A

| | | Signature | | | | | | | | |
|---|---|---|---|---|---|---|---|---|---|---|
| | | derivation | | Signature | Sensitivity | | Specificity | | AUC | |
| # | Algorithm | method | # Filtered | Size | Mean | SE | Mean | SE | Mean | SE |
| 1 | Neural | RF.imp | 75 | 11 | 72.00% | 2.00% | 71.33% | 2.99% | 71.67% | 1.27% |
| | Network | | | | | | | | | |
| 2 | Random | Simulated | 200 | 15 | 60.00% | 1.49% | 80.67% | 2.10% | 70.33% | 0.96% |
| | Forest | Annealing | | | | | | | | |

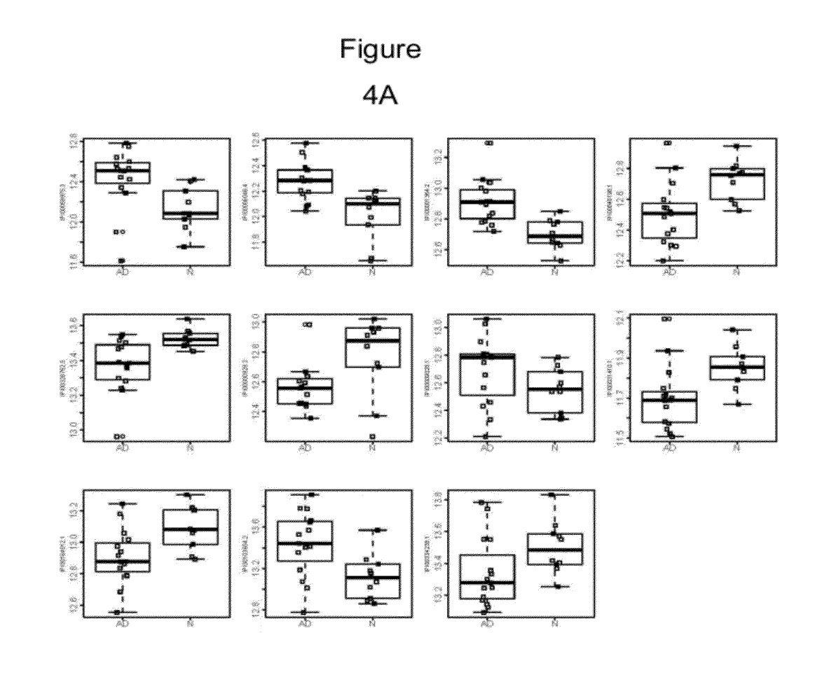

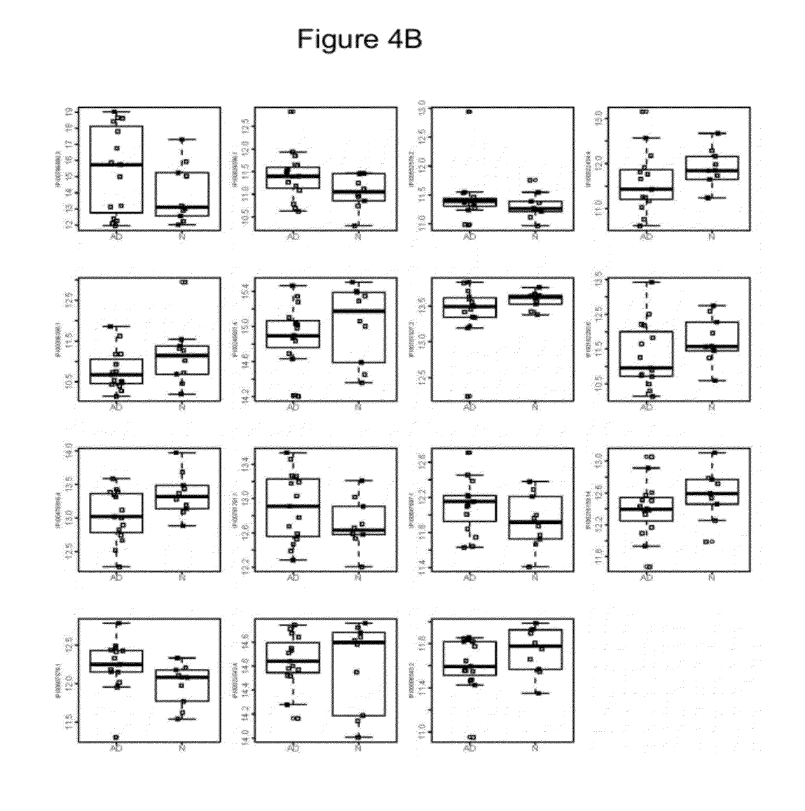

The first signature summarized in Table 3A was derived by first filtering out the top-75 proteins using a robust version of t-statistic, then selecting the 11 best proteins based on the relative importance scores of the Random Forests algorithm, followed by the application of the Neural Network model on these 11 proteins to classify AD and Control subjects. This optimal signature of 11 proteins (Table 3B), had a sensitivity and specificity of 72% (5E=2%) and 71.33% (SE=2.99%), respectively, for classifying CSF from AD subjects versus age-matched controls. The second signature was derived by first filtering out the top-200 proteins using a robust version of t-statistic, then selecting the 15 best proteins based on the Simulated Annealing algorithm, followed by the application of the Random Forests model on these 15 proteins to classify AD and Control subjects. This optimal signature of 15 proteins (Table 3C) had a specificity and sensitivity of 60% (SE=1.49%) and 80.67% (SE=2.1%), respectively, for classifying CSF from AD subjects versus age-matched controls. See FIGS. 4A & 4B for the graphs of individual proteins in these two signatures.

TABLE 3B

| # | Protein ID | Annotation |
|---|---|---|
| 1 | IPI00009028.1 | _Tetranectin |
| 2 | IPI00334238.1 | _neuronal_pentraxin_receptor |
| 3 | IPI00000828.3 | _Proenkephalin_A |
| 4 | IPI00164012.1 | _Actin-like_protein_7A |
| 5 | IPI00001364.2 | _Isoform_A_of_GC-rich_sequence_DNA-binding_factor_homolog |
| 6 | IPI00103604.2 | _Voltage-dependent_calcium_channel_gamma-8_subunit |
| 7 | IPI00848198.1 | _Conserved_hypothetical_protein |
| 8 | IPI00031410.1 | _FKBP12-rapamycin_complex-associated_protein |
| 9 | IPI00006046.4 | _Zinc_finger_protein_536 |
| 10 | IPI00328762.5 | _Isoform_1_of ATP-binding_cassette_sub-family_A_member 13 |
| 11 | IPI00059975.3 | _Isoform_2_of_Synaptotagmin-like_protein_2 |

Table 3B shows proteins used to generate a signature that identified CSF from AD subjects with a sensitivity of 72% (SE=2%) and specificity of 71.33% (SE=2.99%).

TABLE 3C

| # | Protein ID | Annotation |
|---|---|---|
| 1 | IPI00552578.2 | _Serum_amyloid_A_protein |
| 2 | IPI00022434.4 | _Uncharacterized_protein_ALB |
| 3 | IPI00006543.2 | _Complement_factor_H-related_5 |
| 4 | IPI00101927.2 | _Leucine_zipper_putative_tumor_suppressor_2 |
| 5 | IPI00791761.1 | _9_kDa_protein |
| 6 | IPI00829596.1 | _Protein_KIAA0323 |
| 7 | IPI00182293.6 | _Guanylate_kinase |
| 8 | IPI00216159.1 [symbol] | _Glucosamine--fructose-6-phosphate_aminotransferase_[isomerizing]_2 |
| 9 | IPI00786880.3 | _similar_to_KIAA1783_protein |
| 10 | IPI00607576.1 | _Isoform_1_of_Transmembrane_protein_C9orf5 |
| 11 | IPI00478916.4 | _DNA_cross-link_repair_1A_protein |
| 12 | IPI00022543.4 | _GPI-anchor_transamidase |
| 13 | IPI00006395.1 | _Guanine_nucleotide-binding_protein_G(olf)_subunit_alpha |
| 14 | IPI00847697.1 | _Uncharacterized_protein_C9orf109 |
| 15 | IPI00248651.4 | _Isoform_1_of_DNA_polymerase_zeta_catalytic_subunit |

[symbol]

indicates data missing or illegible when filed

Table 3C shows proteins used to generate a signature that identified CSF from AD subjects with a sensitivity of 60% (SE=1.49%) and specificity of 80.67% (SE=2.1%),

Log-transformed quantile-normalized data from each of the 4072 peptides corresponding to the 892 identified proteins were then analyzed in the same manner as described in detail above for the proteins to identify optimal peptide signatures that provide a robust classification between AD and Control subjects.

Two of the best signatures are summarized in Table 4A below.

TABLE 4A

| | | Signature | | Signature | Sensitivity | | Specificity | | AUC | |
|---|---|---|---|---|---|---|---|---|---|---|
| # | Algorithm | derivation | # Filtered | Size | Mean | SE | Mean | SE | Mean | SE |
| 1 | Neural | RF.imp | 300 | 6 | 78.00% | 4.27% | 90.67% | 2.68% | 84.33% | 2.12% |
| | Network | | | | | | | | | |
| 2 | Neural | RF.imp | 500 | 8 | 76.00% | 2.91% | 90.00% | 2.47% | 83.00% | 2.11% |
| | Network | | | | | | | | | |

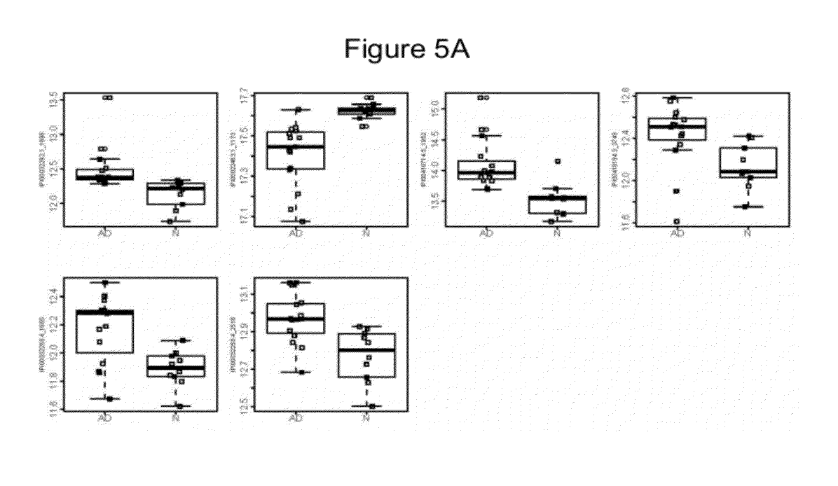

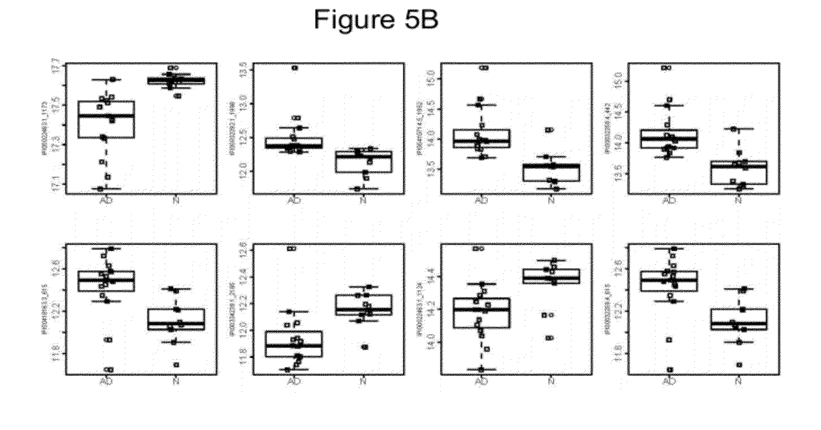

The first signature was derived by first filtering out the top-300 peptides using a robust version of t-statistic, then selecting the 6 best peptides based on the relative importance scores of the Random Forests algorithm, followed by the application of the Neural Network model on these 6 peptides to classify AD and Control subjects. This optimal signature of 6 peptides (Table 4B), had a sensitivity and specificity of 78% (SE=4.27%) and 90.67% (SE=2.68%) respectively, for classifying CSF from AD subjects versus age-matched controls. The second signature was derived by first filtering out the top-500 peptides using a robust version of t-statistic, and then selecting the 8 best peptides based on the relative importance scores of the Random Forests algorithm, followed by the application of the Neural Network model on these 8 peptides to classify AD and Control subjects. This optimal signature of 8 peptides (Table 3C) had a specificity and sensitivity of 76% (SE=2.91%) and 90% (SE=2.47%) respectively, for classifying CSF from AD subjects versus age-matched controls. See FIGS. 5A & 5B for the graphs of individual peptides in these two signatures.

TABLE 4B

| # | Protein ID | Peptide ID | Sequence | Annotation |
|---|---|---|---|---|
| 1 | IPI00022463.1 | 1173 | KPVDEYKDCHLAQVPSHTVVAR | _Serotransferrin |
| 2 | IPI00032258.4 | 1665 | DDPDAPLQPVTPLQLFEGR | _Complement_C4-A |
| 3 | IPI00032258.4 | 2518 | ASAGLLGAHAAAITAY | _Complement_C4-A |
| 4 | IPI00032292.1 | 1998 | LQDGLLHITTCSFVAPWNSLSLAQR | _Metalloproteinase_inhibitor_1 |
| 5 | IPI00410714.5 | 1952 | KVADALTNAVAHVDDMPNALSALSDLHAHK | _Hemoglobin_subunit_alpha |
| 6 | IPI00418194.3 | 3749 | PSSLTNLSSSSGMTSLSSVSGSVMSV | _Breast_cancer-associated_antigen_SGA-72M |

Table 4B shows the peptides used to generate a signature that identified CSF from AD subjects with a sensitivity and specificity of 78% (SE=4.27%) and 90.67% (SE=2.68%) respectively.

TABLE 4C

| # | Protein ID | Peptide ID | Sequence | Annotation |
|---|---|---|---|---|
| 1 | IPI00022463.1 | 1124 | KSASDLTWDNLK | _Serotransferrin |
| 2 | IPI00022463.1 | 1173 | KPVDEYKDCHLAQVPSHTVVAR | _Serotransferrin |
| 3 | IPI00032258.4 | 442 | TTNIQGINLLFSSR | _Complement_C4-A |
| 4 | IPI00032258.4 | 615 | VTASDPLDTLGSEGALSPGGVASLLR | _Complement_C4-A |
| 5 | IPI00032292.1 | 1998 | LQDGLLHITTCSFVAPWNSLSLAQR | _Metalloproteinase_inhibitor_1 |
| 6 | IPI00334238.1 | 2185 | DGPWDSPALILELEDAVR | _neuronal_pentraxin_receptor |
| 7 | IPI00410714.5 | 1952 | KVADALTNAVAHVDDMPNALSALSDLHAHK | _Hemoglobin_subunit_alpha |
| 8 | IPI00418163.3 | 615 | VTASDPLDTLGSEGALSPGGVASLLR | _complement_component_4B_preproprotein |

Table 4C shows the peptides used to generate a signature that identified CSF from AD subjects with a sensitivity and specificity of 76% (SE=2.91%) and 90% (SE=2.47%) respectively.

### Example 2

### Protein Ranking

Data from proteins as being differentially expressed between control and AD groups as described in Example 1 were further analyzed. Briefly, based on a review of the literature relevant to the known relationships between candidate proteins and the biology of AD, candidate proteins were ranked based on a combination of significant fold-change (>20% increase or decrease), confidence in the detection described in Example 1, and biological relevance to AD. Then, rather than applying an area under the curve analysis as used in Example 1, a measure of protein abundance was generated according to the number of spectra belonging to each protein. Of the proteins that showed different spectral counts, these were cross-correlated to the peptide fold change data obtained in Example 1, although no positive matches were obtained. The raw protein data generated in Example 1 was also “searched” to detect oxidized methionines, in contrast to the methods used in Example 1, which did not do so. Four categories were then chosen and used to narrow down the collective lists of proteins from the original 892 proteins identified in the sample analysis described in Example 1.

Twenty-five (25) proteins were selected for a targeted approach to confirm initial findings, using an MRM method with multiplexed detection using pooled CSF samples from age-matched control or AD subjects. Protein rankings were determined using the following categories: 1) proteins including a peptide that showed the same up or down regulation trend between the initial peptide list and the spectral counts analysis; 2) oxidized methionine-containing peptides; 3) complement proteins (based on several showing more spectral counts in AD than in control); and 4) proteins identified according to the analysis in Example 1 that were not detected by the spectral counts or oxidized methionine analyses but were deemed to have a biological connection to the AD disease state based on reports in the literature.

A two group Analysis of Variance (ANOVA) was done for each protein. This is equivalent to the two group t-test. All transitions for each peptides were averaged for each sample on the log2(AUC) scale to get a single number for protein expression for each sample. The analysis was done in JMP version 8.

Sample preparation was substantially as described above in Example 1. CSF samples from 7 AD subjects or 7 age-matched controls (different from the CSF samples used in Example 1) were pooled. Each pooled CSF sample (Alzheimer's disease samples and age-matched normal samples) was aliquoted into 7 tubes. Albumin and IgG were removed from the sample using Sigma Proteoprep spin columns. Resulting flow through fractions were denatured by 8 M urea, reduced by triethylphosphine, alkylated by iodoethanol, and digested by trypsin. The resulting peptides were separated by a Surveyor HPLC system coupled to a Thermo LTQ mass spectrometer which recorded the mass to charge ratios (m/z) of intact and fragment ions. All of the injections were randomized and the instrument was operated by the same operator for this study.

ABI 4000Qtrap and Dionex Ultimate 3000 HPLC system were used for all injections. For quantitative protein analysis by MRM, an ABI/Sciex 4000 QTRAP hybrid triple quadrupole linear ion trap mass spectrometer (Applied Biosystems) was interfaced with a nanospray source. Source temperature was set at 100° C., and source voltage was set at 2400 V. Collision energy (CE) and declustering potential (DP) for each transition were automatically calculated by the Skyline algorithm. For quantitative measurement, the area under the curve (AUC) was calculated for all transitions using the Skyline algorithm. Peptide identification and quantification was performed as described above.

More specifically, as an alternative to AUC quantitation, the data was analyzed by spectral counting using the number of unique spectra per protein as the metric. Ninety .mzXML files representing the complete set of raw data were made available, and each file was renamed to start with the protein ID number v13082. Each file was also labeled according to “patient number replicate number” such that Alzheimer's patients were identified as S01_{—}01, S01_{—}02, S01_{—}03, S02_{—}01, S02_{—}02, etc. Control samples were named in the same way except using “C” for control rather than “S” (for sample). For compatibility with the Mascot search engine, the .mzXML files were converted to .mgf files using a free program called MZXML2MGF (developed by Hua Xu of the University of Illinois at Chicago). The data was then searched against the human IPI database using Mascot and the following parameters: trypsin cleavage at both ends of the peptide, variable 1 ox methionine, 1 allowed internal missed cleavage (MC), and fixed +44 for cysteine alkylation by iodoethanol. The Mascot protein identification results (equaling 219 proteins) were imported into Scaffold (version 2.5) for comparison of unique spectra recorded per protein per condition.

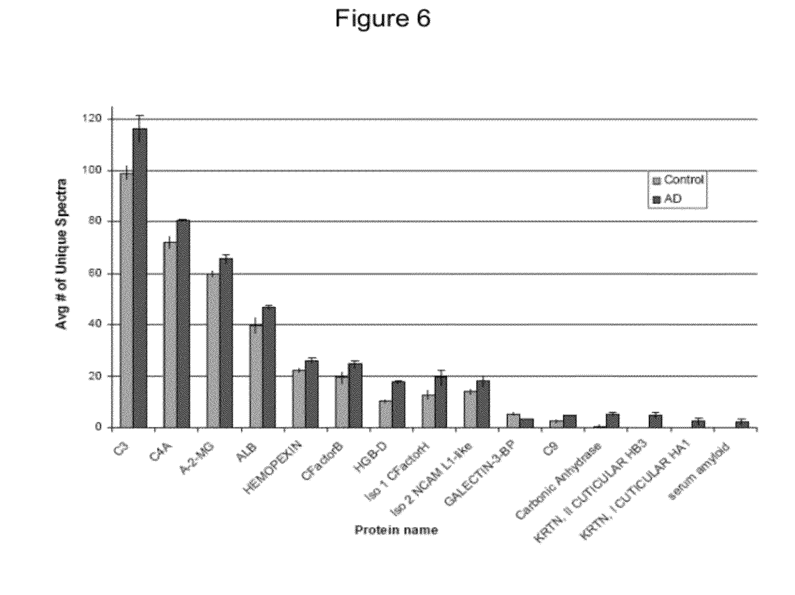

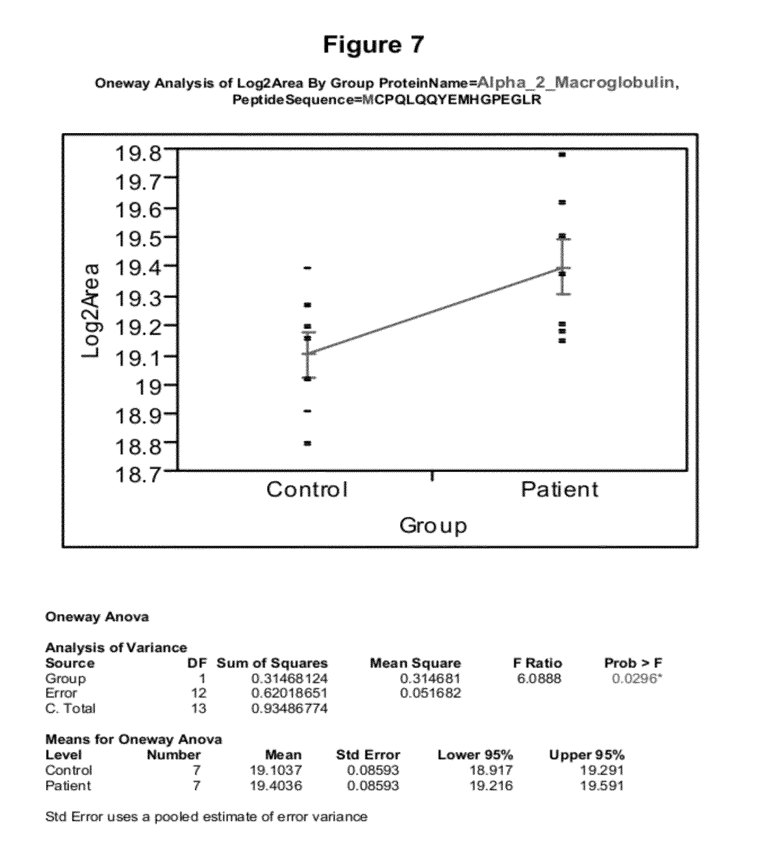

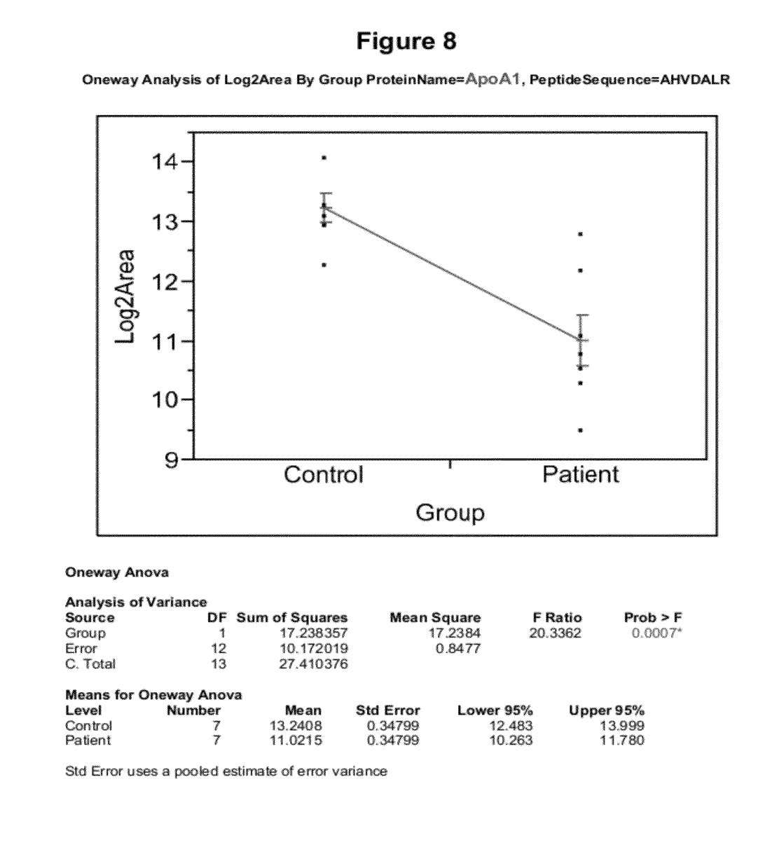

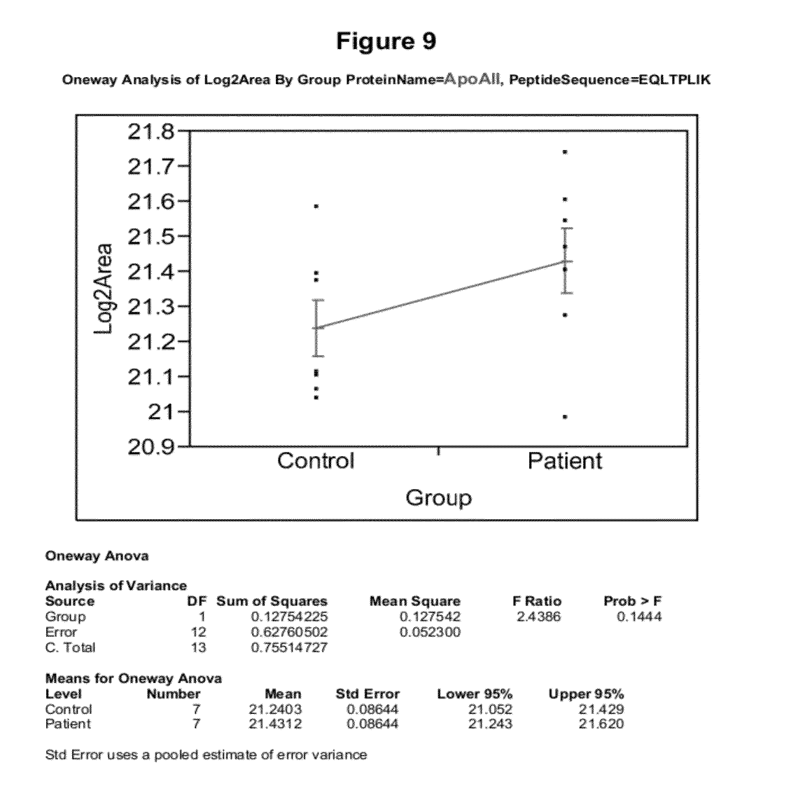

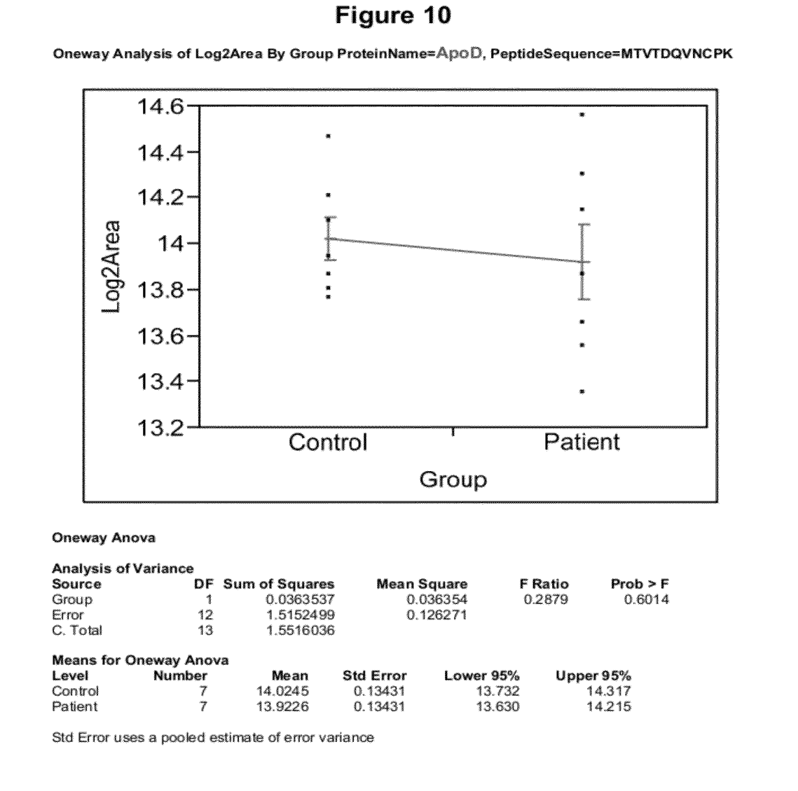

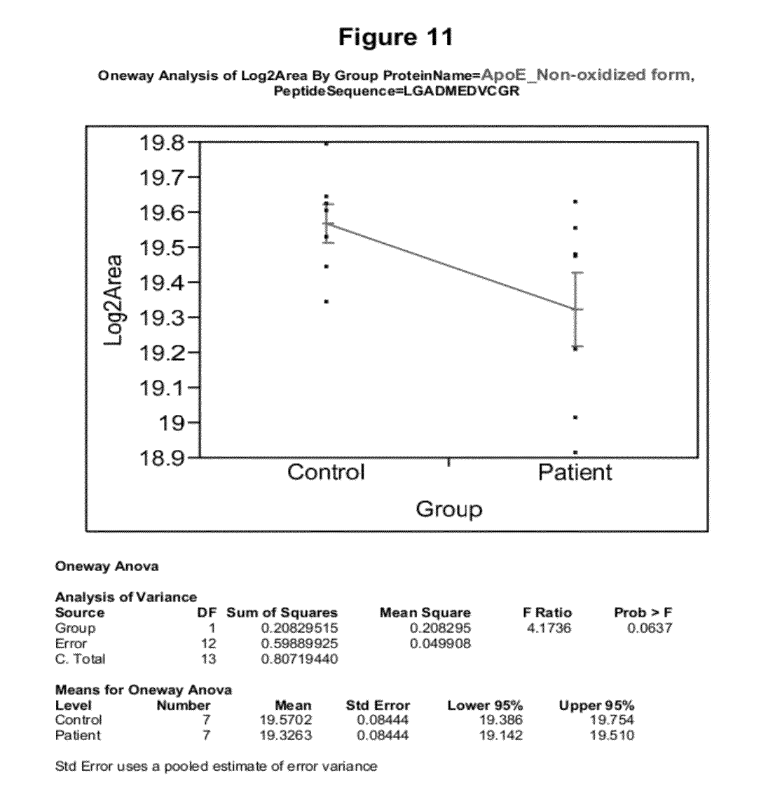

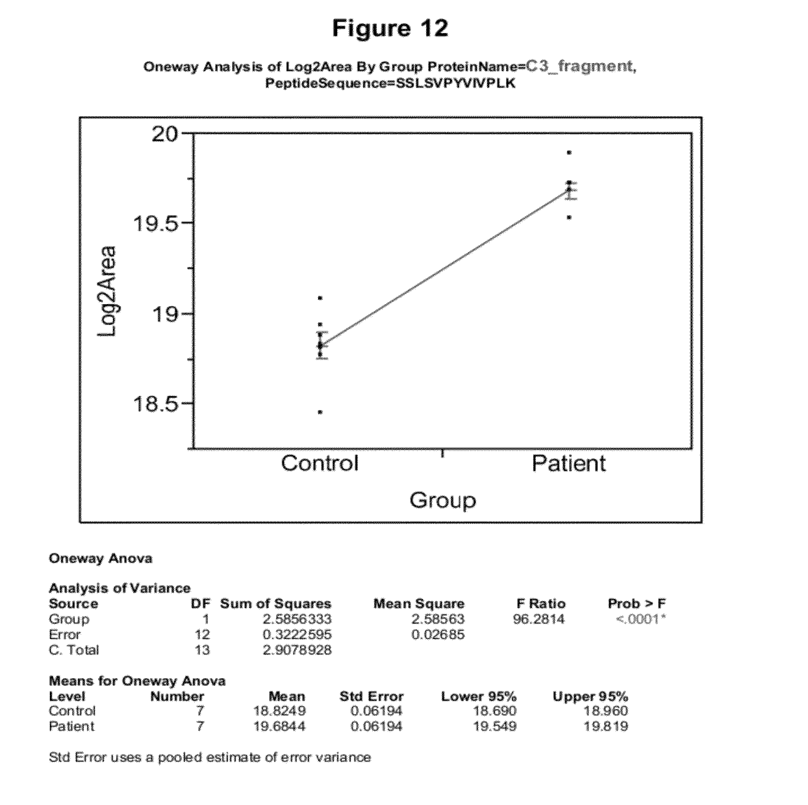

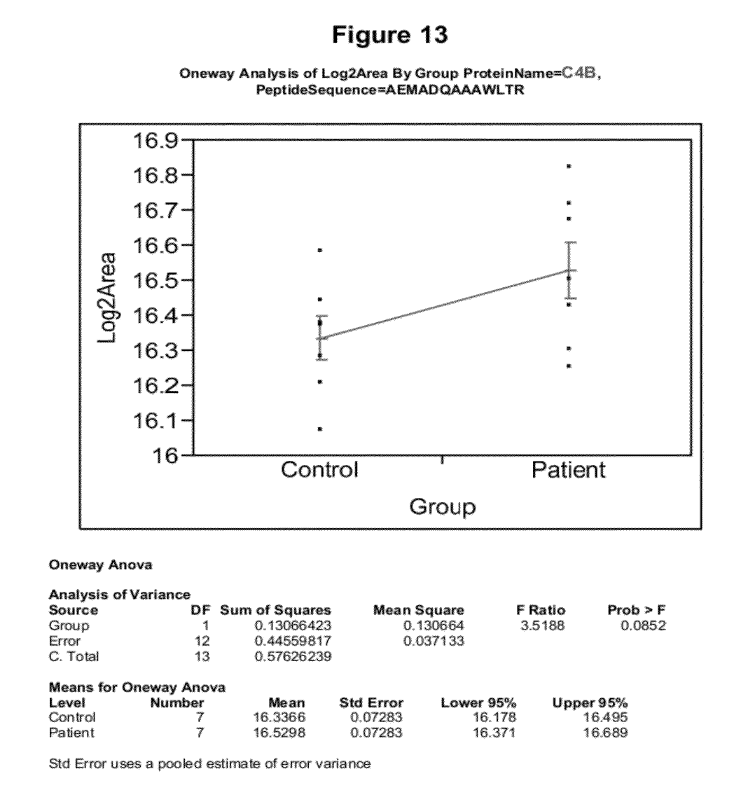

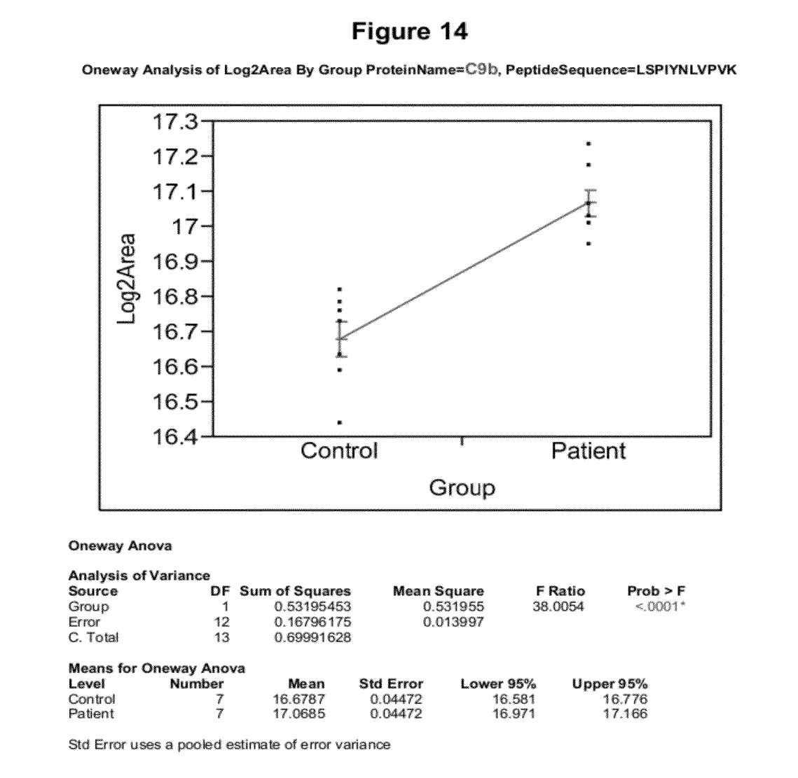

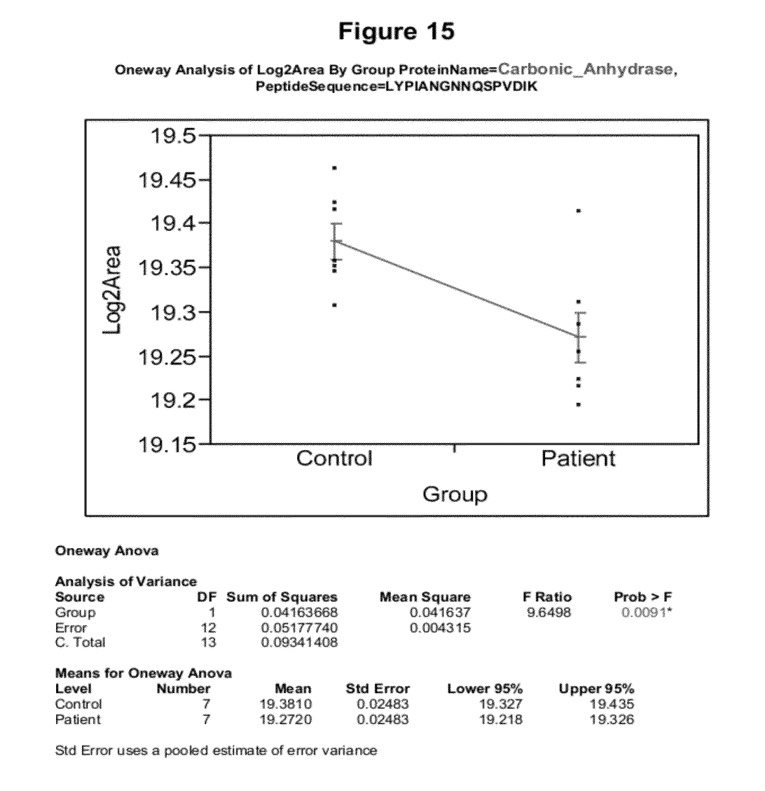

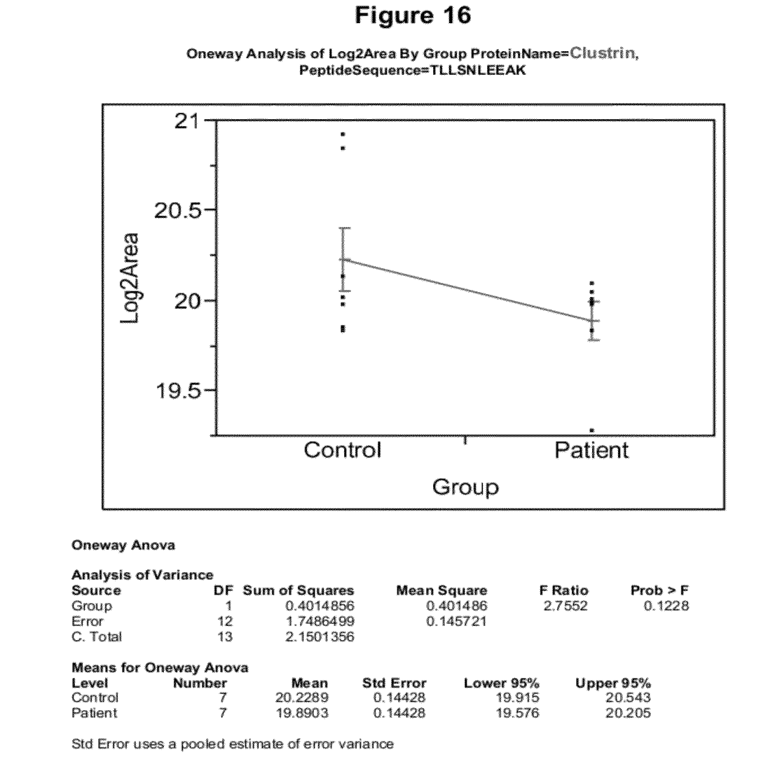

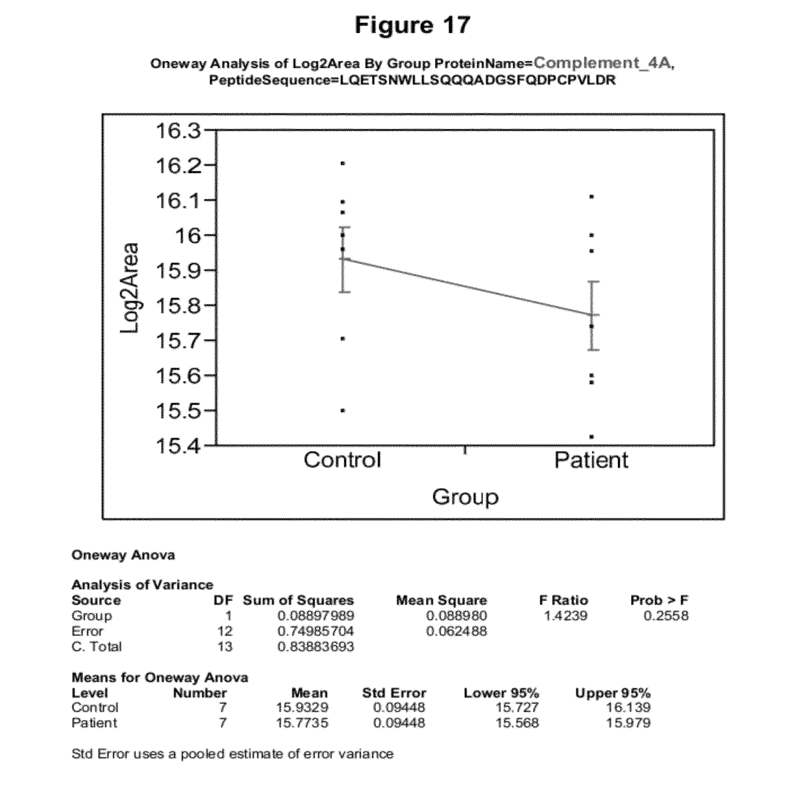

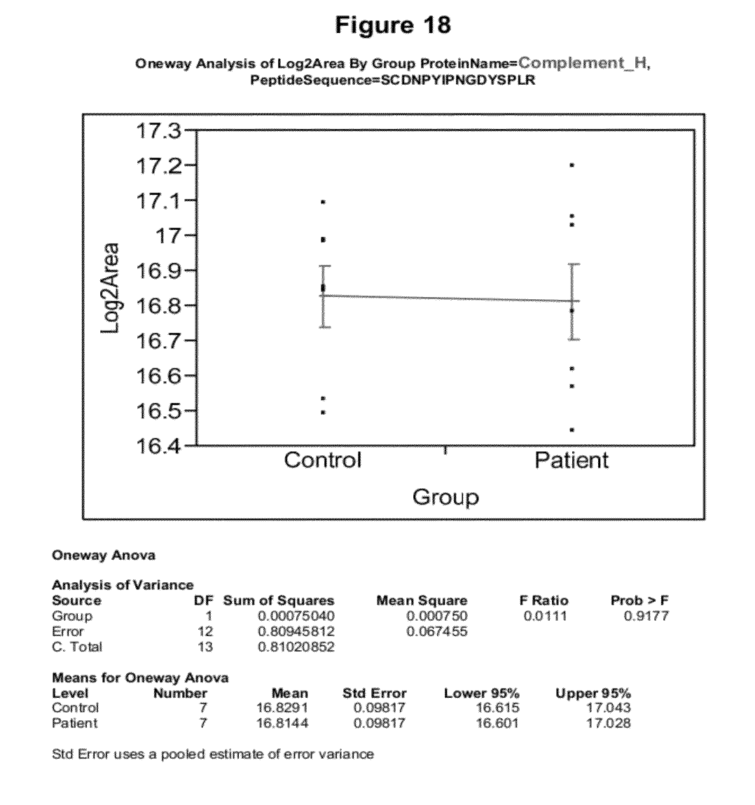

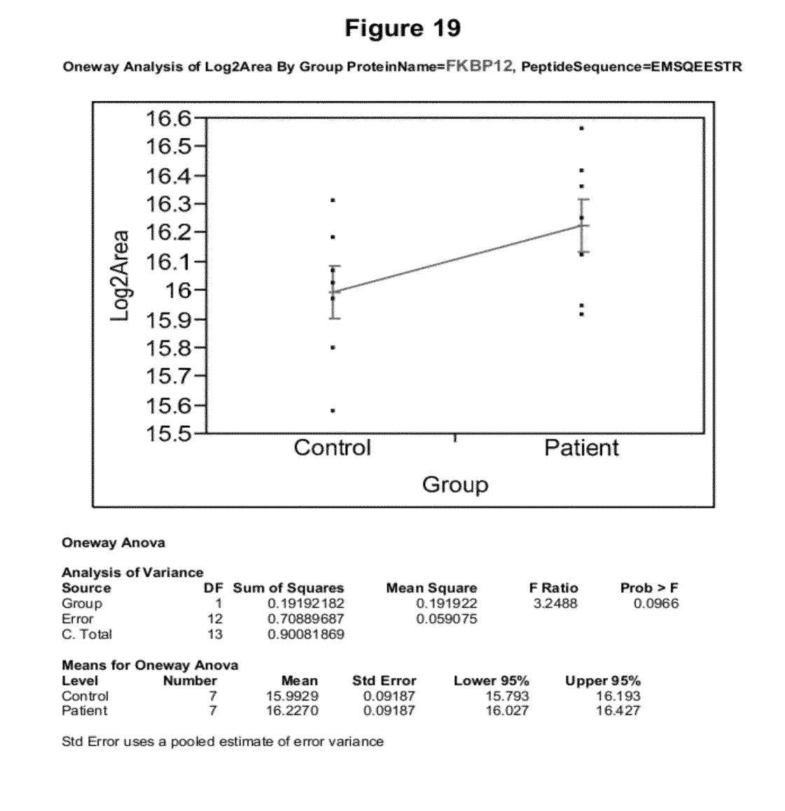

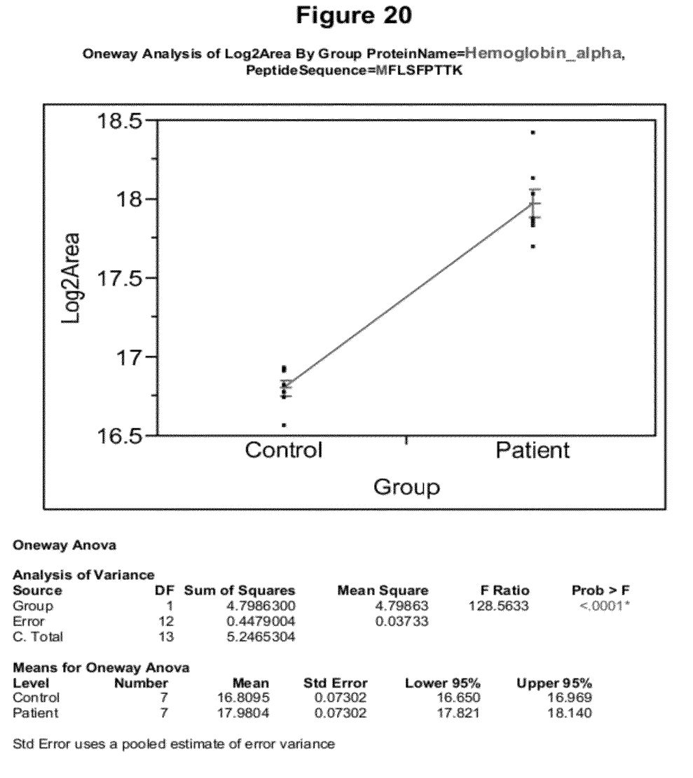

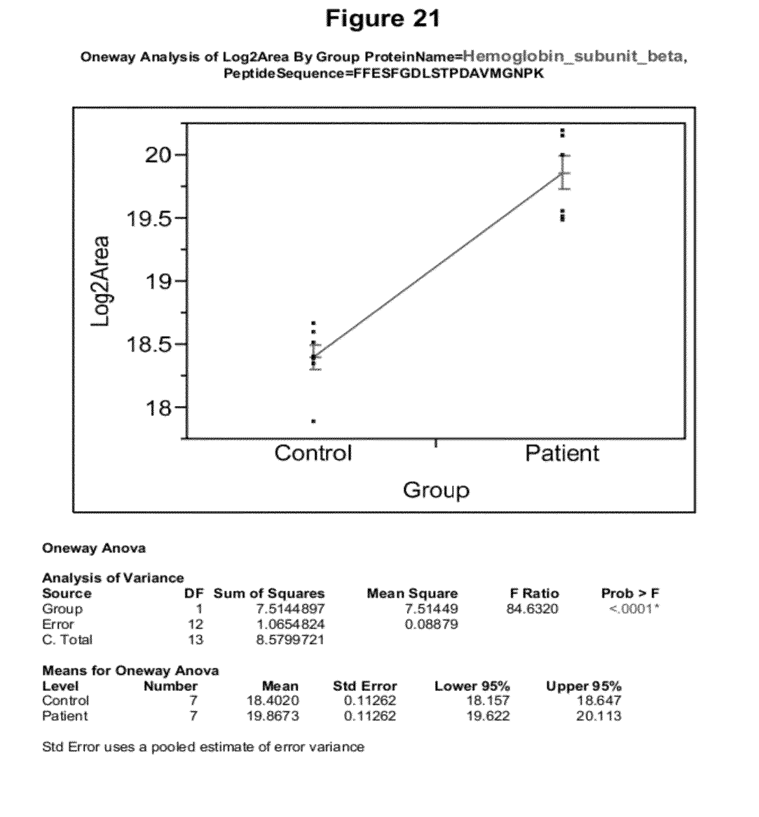

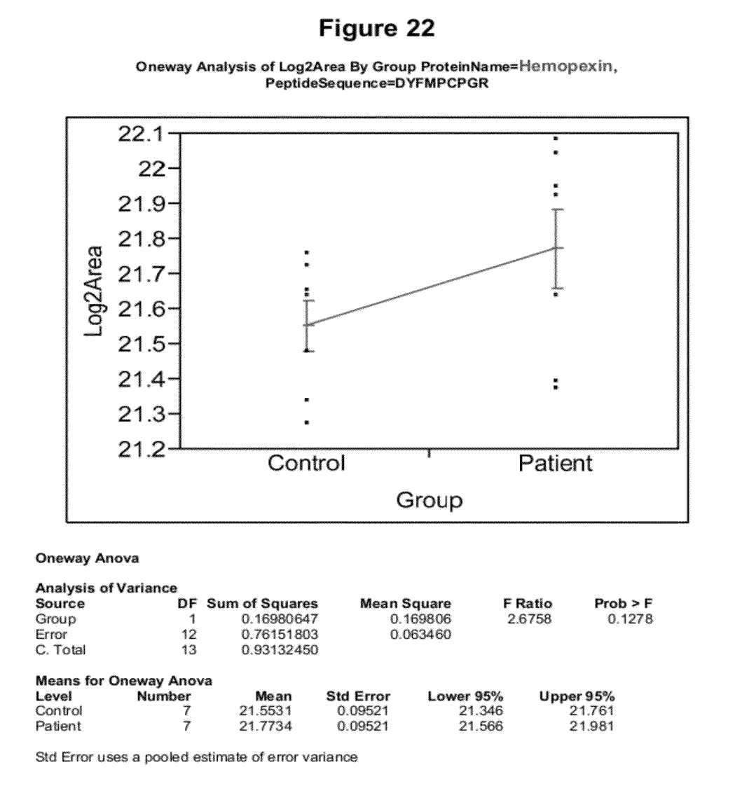

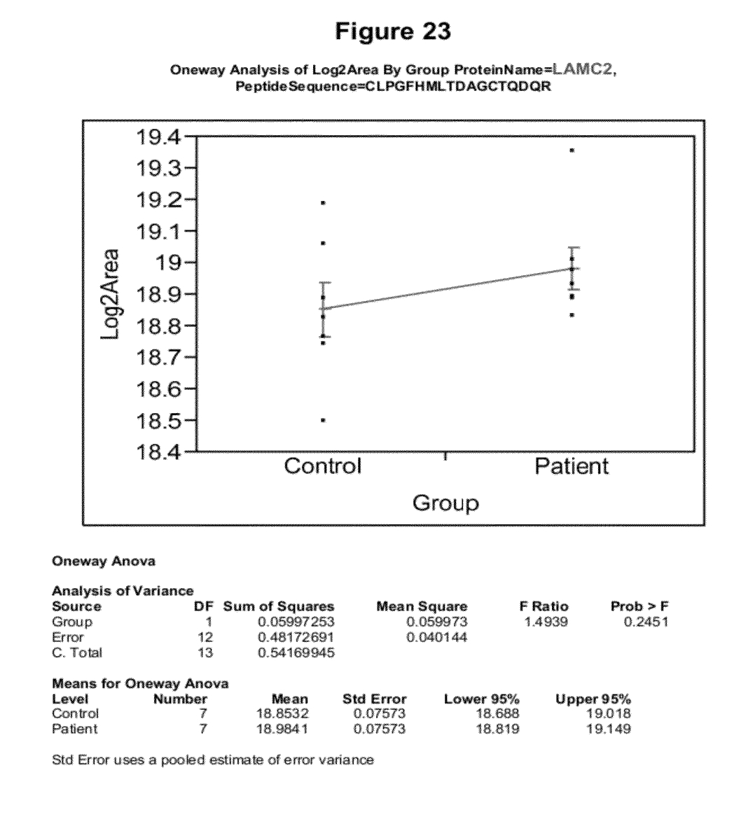

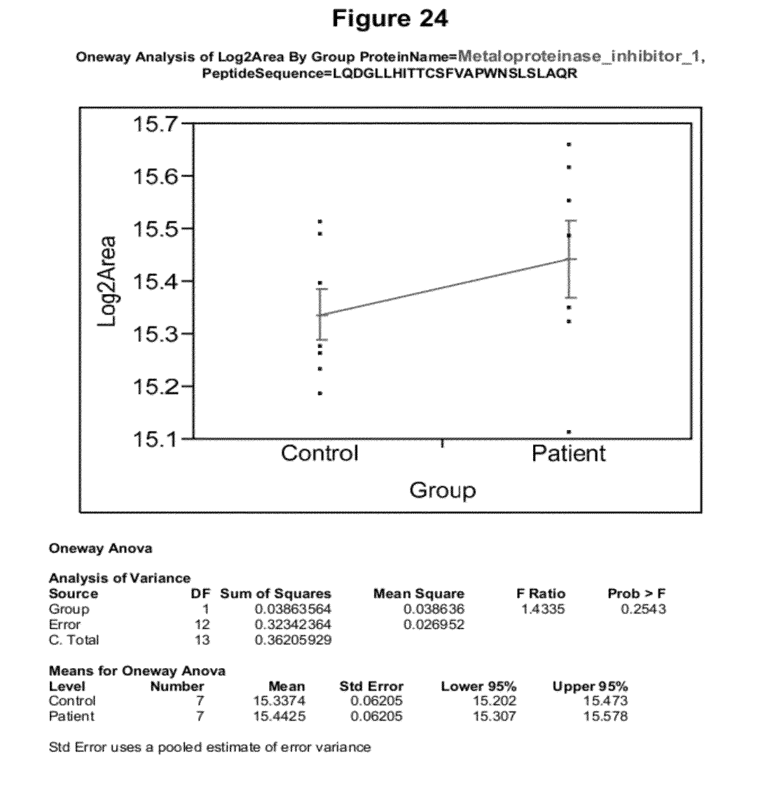

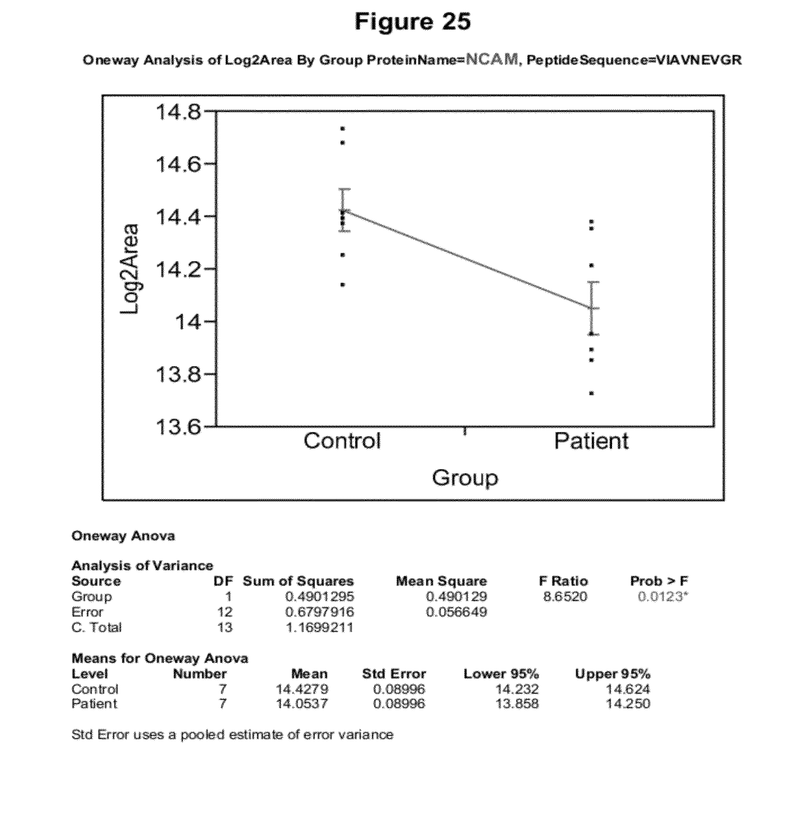

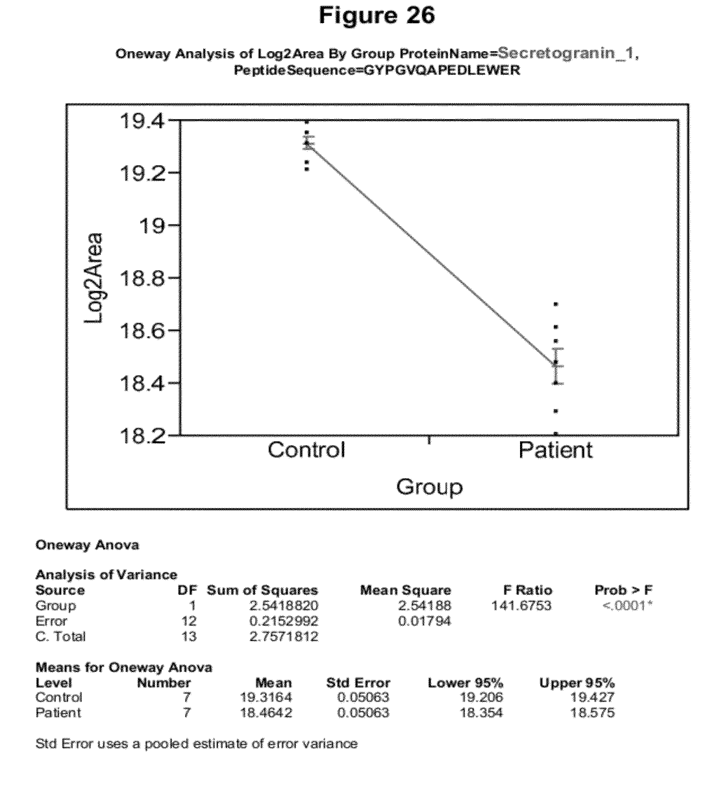

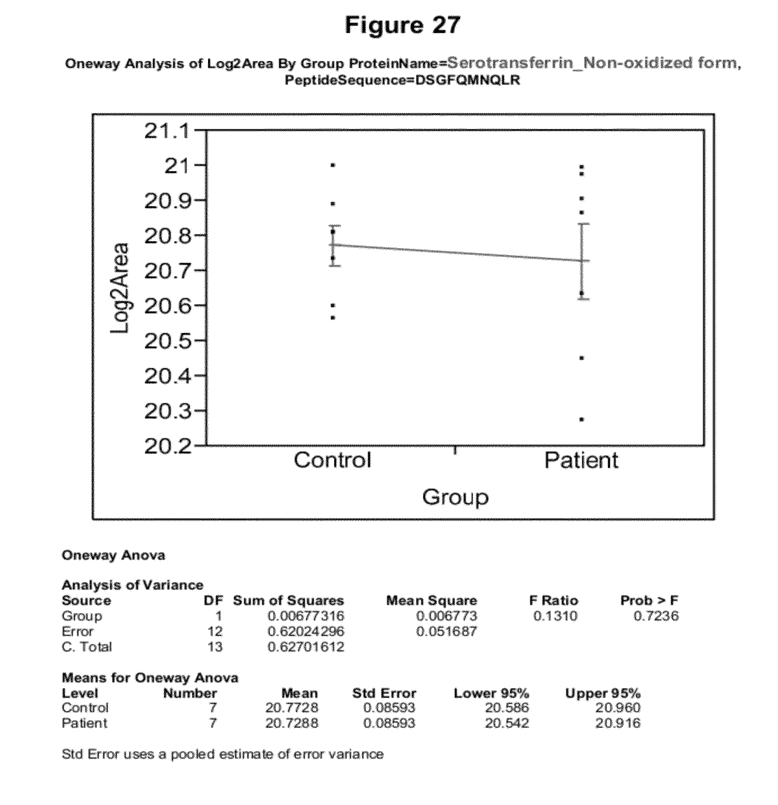

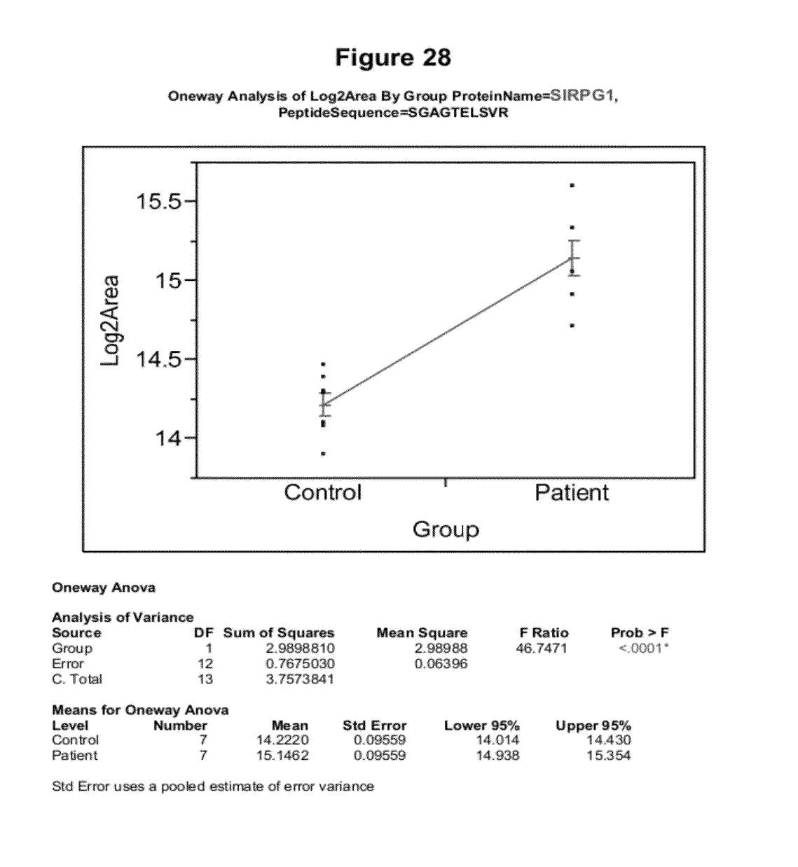

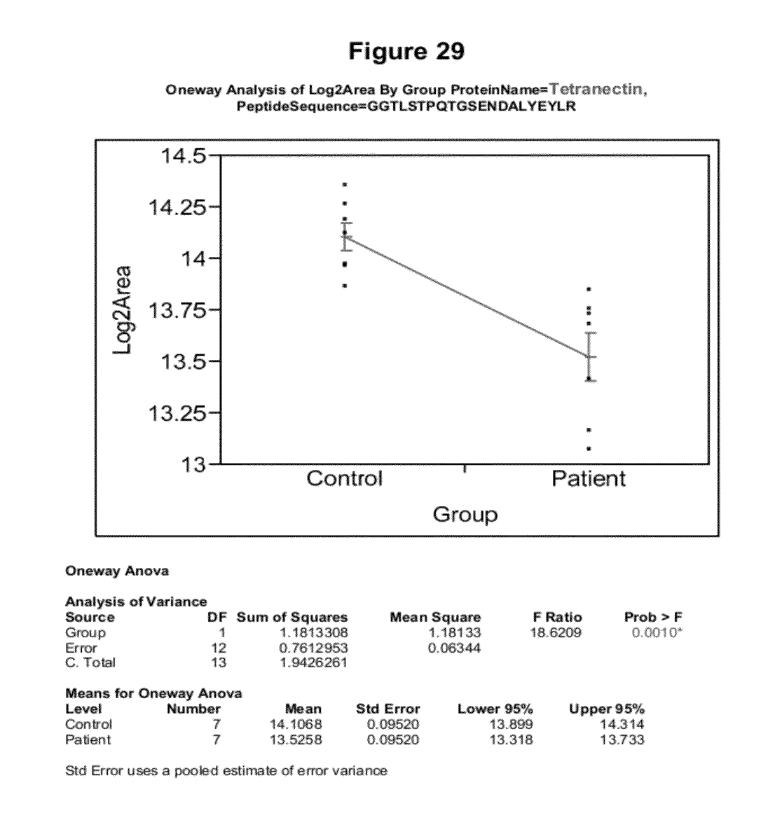

Only those proteins with identifications of 95% probability or greater were considered for evaluation. The number of unique spectra per protein was averaged across replicates and the standard deviation was calculated in Excel. Twenty-five (25) proteins that demonstrated average number of unique spectra with nonoverlapping error bars between AD and control samples are reported in Table 5. A bar chart is also presented in FIG. 6 to better visually illustrate these results, for a subset of fifteen of the proteins set forth in Table 5, which is a complete list of the CSF proteins and peptides identified by the above spectral counts analysis which all demonstrated average number of unique spectra with nonoverlapping error bars between AD and control samples. Thus, these 25 proteins were confirmed as biomarkers for AD and may be useful as markers for other neurological disorders. FIGS. 7-29 show results for twenty-three of these individual proteins in pooled CSF samples from age-matched control (Control) or AD (Patient) subjects.

TABLE 5

| | | | | | | | Control | AD (# | | | | |
|---|---|---|---|---|---|---|---|---|---|---|---|---|
| | | | | | | | (# Unique | Unique | | | | |
| | SEQ. | not in | | | | | Spectra | Spectra | Change | Change | | |
| | ID. | original | | | | | for | for | (Phase | (Phase | Prob > | |
| | NO. | 65 | Protein_ID | Best_Sequence | FoldChange | Annotation | protein) | protein) | I) | II) | F? | Agreement? |
| 1 | 111 | | IPI00654755 | FFESFGDLSTPDAVM*GNPK | Not considered | Hemoglobin subunit beta | 0.0 | 1.0 | ↑ | ND | | |
| 2 | 62 | | IPI00654755 | FFESFGDLSTPDAVMGNPK | 1.75 | Hemoglobin subunit beta | 9.0 | 10.0 | ↑ | ↑ | Y | Y |
| 3 | 112 | | IPI00478003 | M*CPQLQQYEMHGPEGLR | | | | | | | | |
| | | | | (note second | NA | Alpha-2-macroglobulin | 60.0 | 65.7 | ↑ | ↑ | Y | Y |
| | | | | unoxidized | | | | | | | | |
| | | | | methionine) | | | | | | | | |
| 4 | 37 | | IPI00418163 | AEMADQAAAWLTR | 1.26 | Complement_C4-B | ? | ? | ↑ | ↑ | N | Y |
| | | | | (Unique to C4B | | | | | | | | |
| | | | | variant) | | | | | | | | |
| 5 | 113 | | IPI00410714 | MFLSFPTTK | NA | Hemoglobin subunit alpha | 10.3 | 13.3 | ↑ | data not shown | | |
| 6 | 114 | | IPI00410714 | M*FLSFPTTK | Not considered | Hemoglobin subunit alpha | 0.0 | 1.0 | ↑ | ↑ | Y | Y |
| 7 | 115 | Y | IPI00299059 | VIAVNEVGR | NA | Neural Cell Adhesion | 14.0 | 18.0 | ↑ | ↓ | Y | N |
| | | | | (Fibronectin | | Molecule L1-Like Protein | | | | | | |
| | | | | type III 1) | | | | | | | | |
| 8 | 116 | | IPI00291262 | TLLSNLEEAK | −1.24 | Clusterin | 23.7 | 24.7 | ↓ | ↓ | N | Y |
| 9 | 105 | | IPI00291262 | TLLSNLEEAKK (need to | −1.24 | Clusterin | 23.7 | 24.7 | ↓ | ↓ | N | Y |
| | | | | target +/− K) | | | | | | | | |
| 10 | 77 | | IPI00215983 | LYPIANGNNQSPVDIK | 1.66 | Carbonic Anhydrase | 0.3 | 5.3 | ↑ | ↓ | Y | N |
| 11 | 66 | same | | | | | | | | | | |
| | | name, | | | | | | | | | | |
| | | different | IPI00164623 | SSLSVPYVIVPLK | 1.18 | Complement C3 | 99.0 | 116.0 | ↑ | ↑ | Y | Y |
| | | IPI #'s | | | | | | | | | | |
| 12 | 83 | | IPI00032292 | LQDGLLHITTCSFVAPWNSLS | 1.28 | Metalloproteinase_ | 1.0 | 2.3 | ↑ | ↑ | N | Y |
| | | | | LAQR | | inhibitor_1 | | | | | | |
| 13 | 14 | | IPI00032258 | LQETSNWLLSQQQADGSFQ | 1.72 | Complement_C4-A | 72.0 | 80.7 | ↑ | ↓ | N | N |
| | | | | DPCPVLDR (Unique to | | | | | | | | |
| | | | | C4A variant) | | | | | | | | |
| 14 | 117 | | IPI00031410 | EMSQEESTR (288KDa, | −1.11 | FKBP12-rapamycin | ND | ND | ↓ | ↑ | N | N |
| | | | | long-shot) | | complex-associated protein | | | | | | |
| 15 | 118 | Y | IPI00029739 | SCDNPYIPNGDYSPLR | NA | Complement H | 12.7 | 19.3 | ↑ | ↓ | N | N |
| | | | | (sushi repeat 5) | | | | | | | | |
| 17 | 98 | | IPI00027721 | QADTTQYVPMLER (kinase | 1.83 | PDGFRA | ND | ND | ↑ | ND | | |
| | | | | domain, no report of | | | | | | | | |
| | | | | phospho-Y) | | | | | | | | |
| 18 | 119 | Y | IPI00022488 | DYFMPCPGR (sequence | NA | Hemopexin | 22.0 | 26.0 | ↑ | ↑ | N | Y |
| | | | | unique to AD) | | | | | | | | |
| 19 | 120 | | IPI00022463 | DSGFQMNQLR | See below** | Serotransferrin | 70.3 | 71.7 | ↑ | ↓ | N | N |
| 20 | 121 | | IPI00022463 | DSGFQM*NQLR | Not considered | Serotransferrin | 2.0 | 2.7 | ↑ | ND | | |
| 21 | 122 | Y | IPI00022395 | LSPIYNLVPVK (specific | NA | Complement 9 | 2.3 | 5.0 | ↑ | ↑ | Y | Y |
| | | | | to C9b product) | | | | | | | | |
| 22 | 123 | Y | IPI00021842 | LGADMEDVCGR | NA | Apolipoprotein E | 25.0 | 25.0 | ↓(?) | ↓ | N | Y |
| 23 | 124 | | IPI00021842 | LGADM*EDVCGR | Ox not | Apolipoprotein E | 0.0 | 1.0 | ↑ | ND | | |
| | | | | | considered | | | | | | | |
| 24 | 82 | | IPI00021841 | AHVDALR | −1.18 | Apolipoprotein A-1 | 25.7 | 25.3 | ↓ | ↓ | Y | Y |
| 25 | 90 | | IPI00015117 | CLPGFHMLTDAGCTQDQR | 1.78 | LAMC2 | ND | ND | ↑ | ↑ | N | Y |
| | | | | (EGF-like 2) | | | | | | | | |
| 26 | 89 | | IPI00009028 | GGTLSTPQTGSENDALYEYL | 1.29 | Tetranectin | 10.0 | 11.0 | ↑ | ↓ | Y | N |
| | | | | R | | | | | | | | |
| 27 | 125 | Y | IPI00006662 | MTVTDQVNCPK | Monarch added | Apoliprotein D | 10.0 | 9.3 | | ↓ | N | Y |
| 28 | 126 | | IPI00006662 | M*TVTDQVNCPK | Ox not | Apoliprotein D | 0.3 | 1.0 | ↑ | ND | | |
| | | | | | considered | | | | | | | |
| 29 | 106 | | IPI00006601 | GYPGVQAPEDLEWER | −3.07 | Secretogranin-1 | 18.7 | 18.0 | ↓ | ↓ | Y | Y |
| 30 | 127 | | | EQLTPLIK | −1.44 | ApoA-II | ? | ? | ↓ | ↑ | N | N |
| 31 | 128 | Y | IPI00552578 | SFFSFLGEAFDGAR | Detected only | Serum Amyloid A2 | 0.0 | 2.0 | ↑ | ND | | |
| | | | | | in AD | | | | | | | |
| 32 | 129 | Y | ? | SGAGTELSVR | Detected only | SIRPG1 | ? | ? | ↑ | ↑ | Y | Y |
| | | | | | in AD | | | | | | | |

Fold

| | Protein_ID | Annotation | change | Sequence |
|---|---|---|---|---|
| ** | ** | _Serotransferrin | −1.16 | KPVDEYKDCHLAQVPSHTVVAR |
| | IPI00022463.1 | | | (SEQ ID NO: 130) |
| | | | −1.27 | EGYYGYTGAFR |
| | | | | (SEQ ID NO: 131) |
| | | | 1.15 | SDNCEDTPEAGYF |
| | | | | (SEQ ID NO: 132) |

| | |
|---|---|
| *** | ***chosen based on Am. J. Clin. Pathol. 129:526-529 publication |

### Example 3

### Identification of Novel Peptide Biomarkers for Alzheimer's Disease

To identify novel peptide biomarker's from the patients suffering from Alzheimer's disease, samples were collected from patients and healthy volunteers and were analyzed. Cerebrospinal fluid (CSF) from 20 patients were obtained from PrecisionMed, Inc. Fifteen patients were diagnosed with Alzheimer's disease (AD) based on the mini-mental state examination (MMSE) scoring system. Five of these patients gave two samples for a total of 20 CSF samples corresponding to the AD group. Ten additional patients were from the age-matched control group (Table 6). Each sample was run in triplicate, which resulted in a total of 90 analyses (Table 7).

TABLE 6
Patient Details

| SUB- | | | | | | CSF | CSF |
|---|---|---|---|---|---|---|---|
| JECT | | GEN- | DIAG- | MMSE | MMSE | 1.0 mL | 1.0 mL |
| ID # | AGE | DER | NOSIS | VISIT 1 | VISIT 2 | VISIT 1 | VISIT 2 |
| 8001 | 83 | M | AD | 17 | 13 | Available | Available |
| 8005 | 80 | M | AD | 17 | 20 | Available | Available |
| 8006 | 91 | M | AD | 22 | 25 | Available | Available |
| 8056 | 75 | M | AD | 15 | 17 | Available | Available |
| 8058 | 72 | F | AD | 15 | 11 | Available | Available |
| 8026 | 78 | F | AD | 14 | N/A | Available | N/A |
| 8037 | 78 | F | AD | 14 | N/A | Available | N/A |
| 8059 | 79 | F | AD | 14 | N/A | Available | N/A |
| 8038 | 76 | M | AD | 15 | N/A | Available | N/A |
| 8007 | 78 | F | AD | 16 | N/A | Available | N/A |
| 8060 | 79 | M | AD | 16 | N/A | Available | N/A |
| 8061 | 82 | F | AD | 16 | N/A | Available | N/A |
| 8064 | 70 | M | AD | 16 | N/A | Available | N/A |
| 8050 | 79 | M | AD | 17 | N/A | Available | N/A |
| 8040 | 87 | M | AD | 19 | N/A | Available | N/A |
| 7856 | 72 | M | Control | N/A | N/A | Available | N/A |
| 7857 | 73 | M | Control | N/A | N/A | Available | N/A |
| 7858 | 76 | M | Control | N/A | N/A | Available | N/A |
| 7860 | 77 | M | Control | N/A | N/A | Available | N/A |
| 7848 | 80 | M | Control | N/A | N/A | Available | N/A |
| 7810 | 81 | M | Control | N/A | N/A | Available | N/A |
| 7815 | 84 | F | Control | N/A | N/A | Available | N/A |
| 7816 | 85 | F | Control | N/A | N/A | Available | N/A |
| 7841 | 89 | F | Control | N/A | N/A | Available | N/A |
| 7811 | 84 | F | Control | N/A | N/A | Available | N/A |

TABLE 7
Sample Analysis Summary

| Condition | # Samples | # Replicates | # Analyses |
|---|---|---|---|
| Control | 10 | 3 | 30 |
| AD | 20 | 3 | 60 |

| | |
|---|---|
| Total Analyses: | 90 |

The data was analyzed by spectral counting using the number of unique spectra per protein as the metric. Ninety .mzXML files representing the complete set of raw data were created. Each file was labeled according to “patient number replicate number” such that Alzheimer's patients were identified as S01_{—}01, S01_{—}02, S01_{—}03, S02_{—}01, S02_{—}02, et. Control samples were named in the same way except using “C” for control rather than “S” (for sample). For compatibility with the Mascot search engine, the .mzXML files were converted to .mgf files using a free program called MZXML2MGF developed by Hua Xu of the University of Illinois at Chicago.

The data was then searched against the human IPI database using Mascot and the following parameters: trypsin cleavage at both ends of the peptide, variable 1 ox methionine (+16), 1 allowed internal missed cleavage (MC), and fixed +44 for cysteine alkylation by iodoethanol. The Mascot protein identification results (equaling 219 proteins) were imported into Scaffold (version 2.5) for comparison of unique spectra recorded per protein per condition.

Only those proteins with identifications of 95% probability or greater were considered for evaluation. The number of unique spectra per protein was averaged across replicates and the standard deviation was calculated in Excel. The average number of unique spectra per protein were then plotted on a bar chart to determine proteins that were detected between conditions with non-overlapping error bars. “Variable” oxidation means that the peptide was expected to be observed with and without an addition of oxygen (+16). Usually if a peptide is oxidized, it exists in both states in a single sample. This data has been provided in Table 5 above. From this analysis following novel peptide biomarkers were identified in the samples of Alzheimer's patients:

| | |
|---|---|
| FFESFGDLSTPDAVM*GNPK | (SEQ ID NO: 111) |
| M*CPQLQQYEMHGPEGLR | (SEQ ID NO: 112) |
| M*FLSFPTTK | (SEQ ID NO: 114) |
| DSGFQM*NQLR | (SEQ ID NO: 121) |
| LGADM*EDVCGR | (SEQ ID NO: 124) |
| M*TVTDQVNCPK | (SEQ ID NO: 126) |

What is claimed is:

1. A method of classifying Alzheimer's disease state of a subject, comprising: a) providing a test sample from the subject; b) determining expression levels in the test sample of at least one protein or peptide biomarker selected from any of the biomarkers set out in TABLES 2A, 2B or 5, or determining expression levels in the test sample of the proteins or peptides comprising any one of the biomarker combinations set out in TABLES 3B, 3C, 4B, or 4C; c) classifying the levels of expression of the selected biomarkers relative to expression levels of the biomarkers in a reference tissue sample as altered or not altered; and d) classifying the test sample according to (c), wherein altered expression levels of the biomarkers in the tissue sample relative to expression levels of the biomarkers in the reference sample indicate a classification of Alzheimer's disease (AD) in the subject.

2. The method of claim 1, wherein the tissue sample comprises a cerebral spinal fluid sample.

3. The method of claim 1, wherein the biomarkers are any one or more of the biomarkers selected from any one of Tables 2A, Table 2B and 5.

4. The method of claim 1, wherein the biomarkers consist of an optimal set of biomarkers as set forth in any one of Tables 3B, 3C, 4B and 4C.

5. A method for classifying Alzheimer's disease (AD) state of a subject, comprising: a) selecting a statistically relevant multi-analyte panel from fluid samples obtained from human subjects including a control cohort consisting of healthy subjects and an AD cohort consisting of subjects diagnosed with AD, in which panel a plurality of protein or peptide biomarkers are differentially expressed to provide expression values for a reference AD panel and a control panel; b) conducting a Random Forests or Simulated Annealing analysis on the multi-analyte data from step (a) to derive a signature; c) applying a classification algorithm to the signature of step (b) to refine the signature; d) obtaining a test fluid sample from the subject; e) determining expression level in the test sample for each of the protein biomarkers used to specify the panel of (a); e) comparing the results of step (e) to the signature obtained from step (c) to obtain an output; and f) determining the classification of the disease state according to the output of step e), wherein the classification is either AD or control.

6. The method of claim 5, wherein the classification algorithm in (c) is selected from: Linear Discriminant Analysis (LDA), Diagonal Linear Discriminant Analysis (DLDA), Diagonal Quadratic Discriminant Analysis (DQDA), Random Forests, Support Vector Machines, Neural Network, and k-Nearest Neighbor method.

7. The method of claim 5, wherein the multi-analyte panel consists of an optimal panel as set forth in Table 3B, and has at least 72% sensitivity and at least 71% specificity for Alzheimer's disease.

8. The method of claim 5, wherein the multi-analyte panel consists of an optimal panel as set forth in Table 3C, and has at least 60% sensitivity and at least 80% specificity for Alzheimer's disease.

9. The method of claim 5, wherein the multi-analyte panel consists of an optimal panel as set forth in Table 4B, and has at least 78% sensitivity and at least 90% specificity for Alzheimer's disease.

10. The method of claim 5, wherein the multi-analyte panel consists of an optimal panel as set forth in Table 4C, and has at least 76% sensitivity and at least 90% specificity for Alzheimer's disease.

11. A computer-implemented method for classifying a test sample obtained from a subject, comprising: (a) obtaining a dataset associated with the test sample, wherein the obtained dataset comprises quantitative data for at least one protein or peptide biomarker selected from any of the biomarkers set out in TABLES 2A, 2B or 5, or the obtained dataset comprises quantitative data for the biomarkers comprising any one of the biomarker combinations as set out in TABLES 3B, 3C, 4B, or 4C; (b) inputting the obtained dataset into an analytical process on a computer that compares the obtained dataset against one or more reference datasets; and (c) classifying the test sample according to the output of the analytical process, wherein the classification is selected from the group consisting of an Alzheimer's disease (AD) classification and a normal classification.

12. The method of claim 11, wherein the test sample is spinal fluid.

13. The method of claim 11, wherein the protein or peptide biomarkers comprise anoptimal panel selected from a multi-analyte panel consisting of any one of the biomarker combinations set out in TABLES 3B, 3C, 4B, or 4C.

14. The method of claim 11, wherein the analytical process comprises applying to the obtained dataset either Random Forests or Simulated Annealing algorithm to derive optimal signatures, and applying at least one algorithm selected from: Linear Discriminant Analysis (LDA), Diagonal Linear Discriminant Analysis (DLDA), Diagonal Quadratic Discriminant Analysis (DQDA), Support Vector Machines, Neural Network, and k-Nearest Neighbor method to fit the classification model on the optimal signatures.

15. A computer system comprising: (a) a database containing information identifying the expression level in spinal fluid of a set of genes encoding at least two proteins or peptide biomarkers set out in any one of TABLES 2A, 2B, 3B, 3C, 4B, 4C and 5; and b) a user interface to view the information.

16. A kit for classifying a test sample obtained from a human subject, comprising reagents for detecting at least one protein or peptide biomarker selected from any one of the biomarkers set out in TABLES 2A, 2B or 5, or reagents for detecting any one of the protein or peptide biomarker combinations as set out in any one of TABLES 3B, 3C, 4B, or 4C.

17. A biomarker indicative of AD selected from any one of Tables 2A, 2B, 3B, 3C, 4B, 4C and 5.

18. An array of primers or probes for classifying one or more test samples for Alzheimer's disease state, the array comprising: at least two different primers or probes coupled to a solid support; wherein each primer or probe is capable of specifically hybridizing under stringent conditions to a protein or peptide biomarker according to claim 17.

19. The array of claim 18, wherein the biomarkers are any one or more biomarkers selected from any of TABLES 2A, 2B and 5 having an altered expression level of each biomarker between the AD disease state and control that is at a q-value of <0.1, or any two or more biomarkers selected from TABLES 2A, 2B and 5 having an altered expression level of each biomarker between the AD disease state and control that is at a p-value of <0.05.

20. An isolated peptide having an amino acid sequence selected from the group consisting of SEQ ID NO: 111, SEQ ID NO: 112, SEQ ID NO: 114, SEQ ID NO: 121, SEQ ID NO: 124, and SEQ ID NO: 126.
